(19) United States
(12) Patent Application Publication
(10) Pub. No.: US 2022/0079522 A1
(43) Pub. Date: Mar. 17, 2022

(54) WEARABLE HEALTH MONITORING HARNESS

(71) Applicants: Hooshang Kaen, Los Angeles, CA (US); James M. Kaen, Los Angeles, CA (US)

(72) Inventors: Hooshang Kaen, Los Angeles, CA (US); James M. Kaen, Los Angeles, CA (US)

(21) Appl. No.: 17/472,686

(22) Filed: Sep. 12, 2021

Related U.S. Application Data
(60) Provisional application No. 63/077,669, filed on Sep. 13, 2020.

(51) Int. Cl. A61B 5/00; A61B 5/113

(52) U.S. Cl. CPC A61B 5/6805; A61B 5/742; A61B 5/1135

(57) ABSTRACT

A wearable health monitoring harness includes a vest, a bus comprising two wires configured to carry digitized signals and power, a plurality of pairs connectors, where each connector in a pair is connected to a wire of the bus, a battery connected to the bus, a set of sensor units, and a controller unit. Each sensor unit comprises sensor circuitry for measuring bodily signals of a person wearing the wearable harness, an analog to digital converter configured to receive analog signals from the sensor circuitry and convert the analog signals to digitized data, a processor configured to communicate data over the bus, and a pair of connector ports for detachably connecting to a pair of the connectors on the bus. The controller unit includes a processor configured to receive digitized data from the set of sensor units processors and send the digitized data to one or more external devices.

### CLAIM OF BENEFIT TO PRIOR APPLICATIONS

This application claims the benefit of U.S. Provisional Patent Application Ser. No. 63/077,669, filed on Sep. 13, 2021. The contents of U.S. Provisional Patent Application 63/077,669 are hereby incorporated by reference.

### BACKGROUND

Electrical signals that are obtained from electrodes attached to the skin may be used to perform diagnostics or monitor health of subjects. Traditionally, the electrodes are placed individually on a subject's body. These individual electrodes may be difficult to handle and may be dislocated with the subject's movement. In order to facilitate the attachment of the electrodes to the subjects' bodies, wearable harnesses or vests have been used. Different electrodes may be mounted on a harness and the subject may wear the harness in order for the electrodes to make contact with the body.

The electrodes, such as the electrocardiogram (ECG) sensors, have to be placed at specific locations on the chest of a patient, usually an area of about one square inch. Since different persons may have different body frame sizes, the traditional harnesses either have to come in several different sizes and/or may need to be adjusted on the subject's body in order to position the electrodes in their specific locations to receive acceptable signals from every electrode.

Keeping different sizes of harnesses may require a large inventory and a person may still need to try several harnesses in order to find a harness that has the right size. Adjustable harnesses may include different types of fasteners that may allow a harness to be fitted on a subject. In either case, when a number of electrodes has to be placed on specific locations on the body, it becomes a difficult task to fit a harness on a person such that every electrode comes to contact with the body at an ideal location. Furthermore, the tangling of wires and the leads connected to the electrodes may be a difficult task to handle.

### BRIEF DESCRIPTION OF THE DRAWINGS

The various embodiments of the present wearable health monitoring harness now will be discussed in detail with an emphasis on highlighting the advantageous features. These embodiments depict the novel and non-obvious wearable health monitoring harness shown in the accompanying drawings, which are for illustrative purposes only. These drawings include the following figures, in which like numerals indicate like parts:

FIG. 1A is a functional diagram illustrating a front view of an example embodiment of a wearable health monitoring harness, according to various aspects of the present disclosure;

FIG. 1B is the back view of the wearable health monitoring harness of FIG. 1A, according to various aspects of the present disclosure;

FIG. 2A is a functional diagram illustrating a front view of an example embodiment of the wearable health monitoring harness of FIGS. 1A-1B, after attaching several sensor units to the wearable harness, according to various aspects of the present disclosure;

FIG. 2B is the back view of the wearable health monitoring harness of FIG. 2A, according to various aspects of the present disclosure;

FIG. 3 is a block diagram illustrating an example breathing rate sensor unit, according to various aspects of the present disclosure;

FIG. 4 is a block diagram illustrating example components of a controller unit of a wearable health monitoring harness, according to various aspects of the present disclosure;

FIG. 5 is a block diagram illustrating example components of a sensor unit used with a wearable health monitoring harness, according to various aspects of the present disclosure;

FIG. 6 is a block diagram illustrating example components of a sensor unit with a wireless communication unit used with a wearable health monitoring harness, according to various aspects of the present disclosure;

FIG. 7 is a block diagram illustrating example components of a sensor unit, with a wireless communication unit and one or more batteries, used with a wearable health monitoring harness, according to various aspects of the present disclosure;

FIG. 8 is a flowchart illustrating an example process for controller of a wearable health monitoring harness to collect and process the sensor units data, according to various aspects of the present disclosure;

FIG. 9 is a flowchart illustrating an example process for sensor unit of a wearable health monitoring harness to collect and/or send data, according to various aspects of the present disclosure;

FIG. 10A is a functional diagram illustrating a front view of an alternative example embodiment of a wearable health monitoring harness, after attaching several sensor units to the wearable harness, according to various aspects of the present disclosure;

FIG. 10B is the back view of the wearable health monitoring of FIG. 10A, according to various aspects of the present disclosure;

FIG. 11A is a functional diagram illustrating a front view of an alternative example embodiment of a wearable health monitoring harness, after attaching several sensor units to the wearable harness, according to various aspects of the present disclosure;

FIG. 11B is the back view of the wearable health monitoring harness of FIG. 11A, according to various aspects of the present disclosure;

FIG. 11C is a functional diagram illustrating a front view of an alternative example embodiment of a wearable health monitoring harness, according to various aspects of the present disclosure.

FIG. 11D is the back view of the wearable health monitoring harness of FIG. 11C, according to various aspects of the present disclosure.

FIG. 12 is a top view of a portion of an example wearable health monitoring harness with a two-wire bus running across the harness, according to various aspects of the present disclosure;

FIG. 13 is top view of a portion of an example wearable health monitoring harness with a two-wire bus and a set of snap connectors for connecting electronic devices to the bus, according to various aspects of the present disclosure;

FIGS. 14A-14C illustrate example electronic devices that may be connected to the connectors on the bus of an example wearable health monitoring harness, according to various aspects of the present disclosure;

FIG. 15 is top view of a portion of an example wearable health monitoring harness with a two-wire bus and a set of clips for connecting electronic devices to the bus, according to various aspects of the present disclosure;

FIG. 16A is a top perspective view of an example clip for connecting the bus of a wearable health monitoring harness to sensors and/or electronic devices that may be attached to the wearable health monitoring harness, according to various aspects of the present disclosure;

FIG. 16B is a side elevation view and FIG. 16C is a top view of the clip of FIG. 16A, according to various aspects of the present disclosure;

FIG. 17 is a top perspective view of another example clip for connecting the bus of a wearable health monitoring harness to sensors and/or electronic devices that may be attached to the wearable health monitoring harness, according to various aspects of the present disclosure;

FIG. 18 is top view of the wearable health monitoring harness of FIG. 15 where the clips are covered by a non-conductive stretchable material, according to various aspects of the present disclosure;

FIG. 19 is a top view of a portion of an example wearable health monitoring harness that includes a bus with stretchable conductors, according to various aspects of the present disclosure;

FIG. 20 is top view of a portion of an example wearable health monitoring harness that includes a bus with stretchable conductors and a set of connectors for connecting electronic devices to the bus, according to various aspects of the present disclosure;

FIG. 21 is top view of a portion of an example wearable health monitoring harness a with bus with stretchable conductors and a set of clips for connecting electronic devices to the bus, according to various aspects of the present disclosure;

FIG. 22 is top view of the wearable health monitoring harness of FIG. 21 where the clips are covered by a non-conductive stretchable material, according to various aspects of the present disclosure;

FIG. 23 is a schematic front view of an audio/video device that may be attached to a wearable health monitoring harness, according to various aspects of the present disclosure;

FIG. 24 is a schematic front view of an electronic device that may receive and display information from the controller of a wearable health monitoring harness, according to various aspects of the present disclosure; and

FIG. 25 is a schematic front view of an electronic device that may receive a warning message from the controller of a wearable health monitoring harness, according to various aspects of the present disclosure.

### DETAILED DESCRIPTION

Wearable harnesses have been used for bringing electrodes in contact with a subject's body. One aspect of the present embodiments includes the realization that the electrodes, such as the ECG sensors, attached to the wearable harnesses has to come in contact with the subject's body on specific areas on the body, typically within an area of approximately one square inch. Depending on the type of the sensor and the body signal being monitored, the area for placing an electrode on the body may be as small as one square inch.

The existing wearable harnesses are provided with the sensors attached to designated locations on the harnesses. Putting a wearable harness on a subject such that all electrodes are attached to the desired areas on the body may, therefore, become a challenging and time consuming task.

In addition, the existing ECG monitoring systems, such as a 12-lead ECG monitoring system, require each electrode to be directly connected to a controller on the harness by a wire. Connecting each electrode to the controller by a separate wire and transferring analog signals from the electrodes to the controller over a long wire may make the signals susceptible to picking up noise and interference. Furthermore, the long wires and the leads connected to the electrodes may tangle.

The present embodiments, as described in detail below, solve the above-mentioned problems by providing a wearable health monitoring harness that allows finetuning the location of every individual electrode on the subject's body once the subject has worn the wearable health monitoring harness. Some embodiments provide sensor units that include sensor circuitry including an electrode, a processor, one or more analog to digital (A/D) converters, and/or memory. The wearable health monitoring harness provides connectors along a bus that may include several wires. The bus may connect the connectors to a controller and/or to one or more batteries attached to the harness. The bus may be used for exchanging control signals, data signals, and/or health status between the controller and the sensor units. The bus may also provide power from the batteries to the sensor units.

The signals received from the sensor circuitry is converted from analog to digital and stored in the sensor unit's memory. The digitized data may then be sent through the bus to the controller. Sending digitized data (instead of analog data) provides the technical advantage of reducing the possibility of noise and interference corrupting the data. In some embodiments, the processor of each sensor unit may further provide error-detection and/or error-correction codes such as, for example, and without limitations, checksum, cyclic redundancy check (CRC), error correcting codes (ECCs), etc., to further enhance the reliability of the data sent from the sensor units.

The controller, in some embodiments, may process the sensor data. The controller may include a communication unit that may transfer the raw and/or processed sensor data to one or more external electronic device through wired and/or wireless connections. The sensor units, in some embodiments, may include a wireless communication unit that may send the digitized data to an external electronic device. In some embodiments, the controller may not be on harness. In these embodiments, the controller may be a mobile device that may wirelessly receive and process the digitized data from the sensor units.

The sensor units may include ECG sensors, body temperature sensors, oxygen sensors, motion sensors, breathing rate sensors, impedance plethysmography sensors, microphones (e.g., for recording lungs' sounds), blood pressure sensors, etc. Some embodiments may attach audio/video devices to the wearable health monitoring harness, for example, to record audio and/or video of the environment, to provide remote communication with persons such as, for example, and without limitations, physicians, nurses, technician, care providers, etc. Some embodiments may attach a display, either as a part of an audio/video device or as a separate unit to the wearable health monitoring harness to provide sensor data, to program the sensor units and/or the controller, to display health status of different devices, such as the controller, the batteries, the sensor units, etc.

The remaining detailed description describes the present embodiments with reference to the drawings. In the drawings, reference numbers label elements of the present embodiments. These reference numbers are reproduced below in connection with the discussion of the corresponding drawing features.

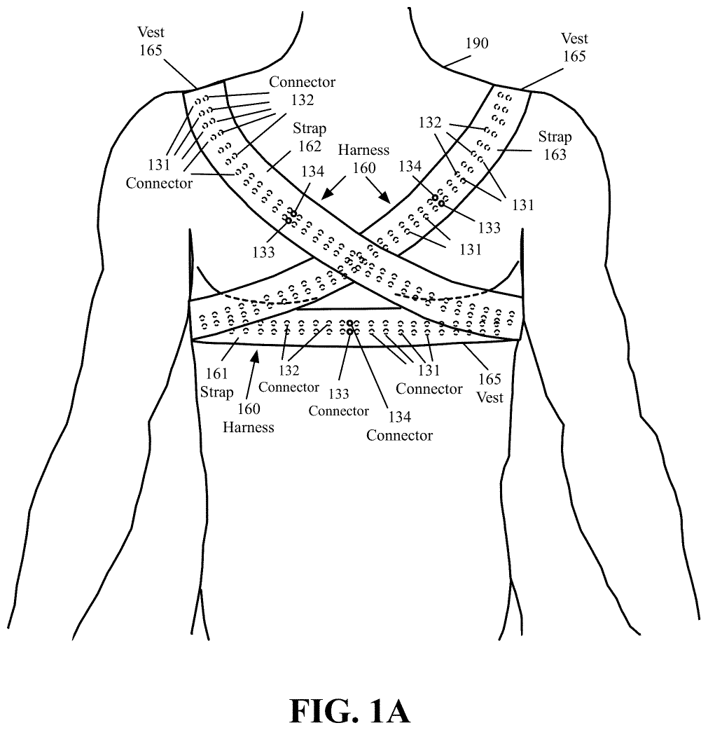

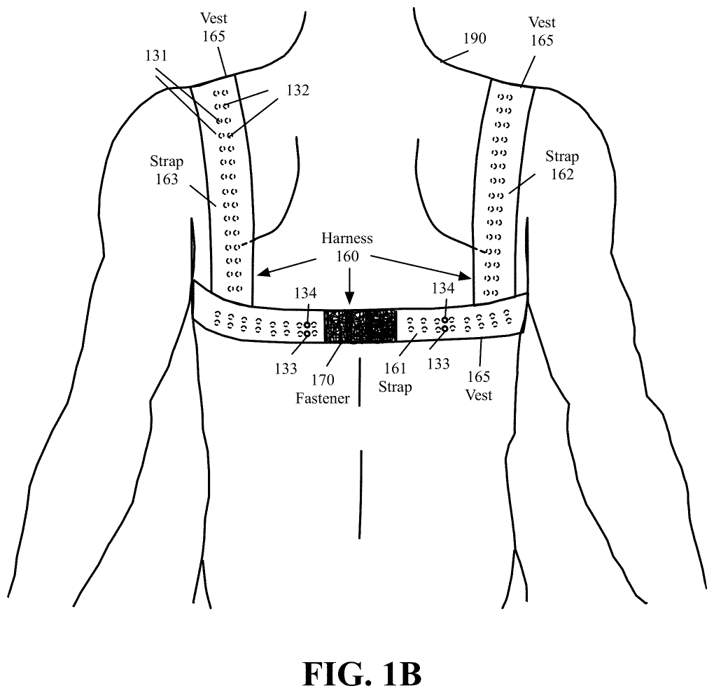

Some embodiments may provide a wearable health monitoring harness for attaching electrodes that monitor different signals on a person's body. FIG. 1A is a functional diagram illustrating a front view of an example embodiment of a wearable health monitoring harness 160, according to various aspects of the present disclosure. FIG. 1B is the back view of the wearable health monitoring harness 160 of FIG. 1A, according to various aspects of the present disclosure.

With reference to FIGS. 1A and 1B, the wearable health monitoring harness 160, in some embodiments, may include a vest 165. The vest 165 in different embodiment may include one or more straps. In the example of FIGS. 1A-1B, the vest 165 may include three straps 161-163. The vest 165 may be made of a stretchable material, such as stretchable fabric, in order to be comfortably worn by a person. The fabric may be, for example, and without limitations, synthetic fiber (e.g., Spandex, Lycra, elastane), synthetic rubber (e.g., Neoprene), etc.

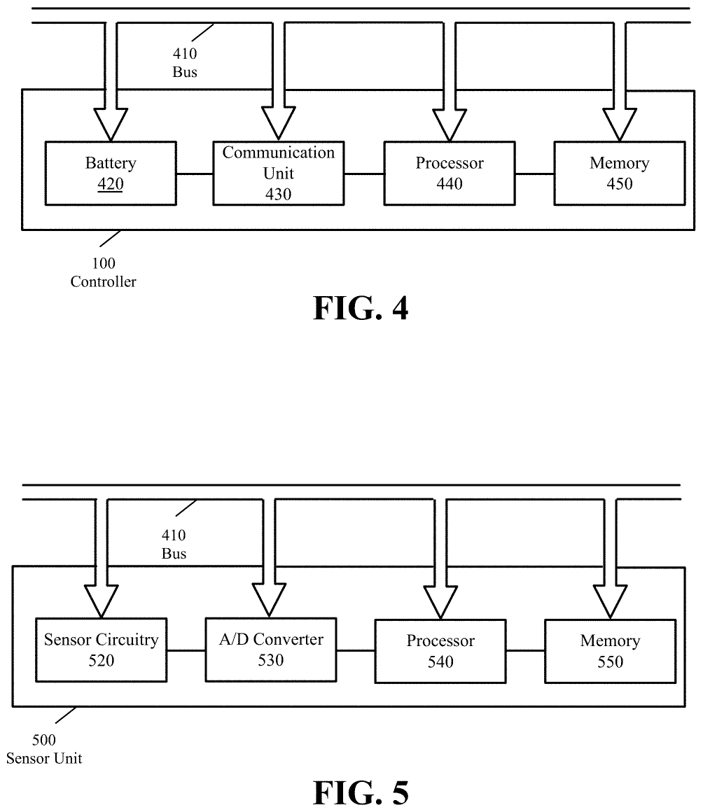

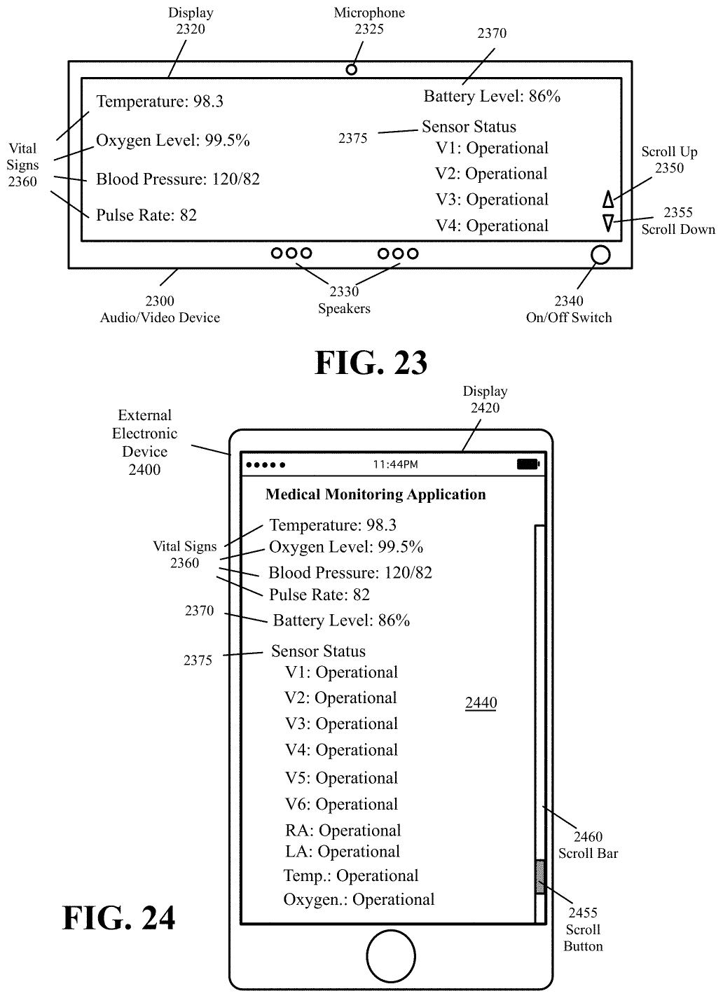

It should be noted that the term vest is herein referred to the portion of the wearable health monitoring harness 160 that is worn by a person 190 and may include the straps 161-163. The term wearable health monitoring harness 160 is referred to the wearable device that in addition to the vest 165 may include a plurality of connectors (such as the connectors 131-134 of FIGS. 1A-1D), a plurality of electronic devices (such as the electronic devices 100-116 of FIGS. 1B-1C), a bus (such as the bus 410 of FIGS. 4-6), one or more audio/video devices (such as the audio/video device 2300 of FIG. 23), and other devices described herein.

As shown, the strap 161 may be worn around the chest, for example below the pectoral line. The two straps 162 and 163 may be worn around the upper body such the two straps 162 and 163 cross each other over the chest of the person 190 and are substantially parallel to each over the back of the person 190. It should be noted that FIG. 1A illustrates the front of the person 190, and FIG. 1B illustrates the back of the person 190.

The strap 161 may be connected to the strap 162 in front and in the back of the vest 165. The strap 161 may also be connected to the strap 163 in front and in the back of the vest 165. The strap 161 may be connected to the straps 162 and 163 by fasteners, by sewing, be snap connectors, by buttons, by stitches, etc. The fasteners, in some embodiments, may be hook-and-loop fasteners that include two components, which may be attached to the opposing surfaces to be fastened. The first component includes tiny hooks and the second component includes small loops. When the two components are pressed together, the hooks may catch in the loops and the two pieces fasten or bind temporarily. An example of the hook-and-loop fasteners is the hook-and-loop fasteners provided by Velcro company.

With reference to FIG. 1B, the strap 161, in some embodiments, may include a fastener 170, such as, for example, and without limitations, a hook-and-loop fastener, a set of buttons, a set of snap connectors, etc., to allow for the vest 165 to be quickly put on or off a person's body. Although in the depicted embodiment the fastener 170 is on the back portion of the strap 161, the fastener 170, in other embodiments, may be placed on the front portion of the strap 161.

With further reference to FIGS. 1A and 1B, the straps 161-163 may include several connectors that may be placed along the length of the straps 161-163. For example, the connectors 131-132 (shown by dashed lines) may be located on the bottom surface of the vest (e.g., on the side of the vest that is facing the person's body) while the correctors 133-134 (shown by solid lines) may be located on the top surface of the vest (e.g., on the side that is away from the person's body). In this disclosure, the terms top surface and bottom surface is used relative to the body of the person who is wearing the vest, with the bottom surface referring to the surface of the vest that touches the body of the person and the top surface referring to the surface of the vest that is away from the body of the person.

As described further below, the connectors 131-134 may be connected to a bus that connects the connectors to one or more batteries and/or to a controller that is located on the harness 160. For example, and without limitations, the connectors 131 and 133 may be connected to a bus wire that may be connected to a positive terminal of the battery (or batteries) and the connectors 132 and 134 may be connected to bus a wire that may be connected to a negative terminal of the battery (or batteries). The negative terminal, in some embodiments, may be a ground terminal and/or may serve as a zero volt and/or a reference terminal.

The connectors 131 and 132 may be used to connect sensor units (e.g., and without limitations, ECG, temperature, oxygen sensors, microphones used for receiving sound from the lungs, etc.) that may have to come in contact with the person's body. The connectors 133 and 134 may be used to connect sensor units (e.g., and without limitations, motion sensors, breathing rate sensors, etc.) and/or electronic devices (e.g., and without limitations, breathing sensors, controller, batteries, audio/video devices, displays, etc.) that may not need to come in contact with the person's body. Since there may be more sensors that have to be in touch with the person's body than the sensors that are connected to the top surface of the harness 160, some embodiments may provide fewer connectors 133-134 than the connectors 131-132.

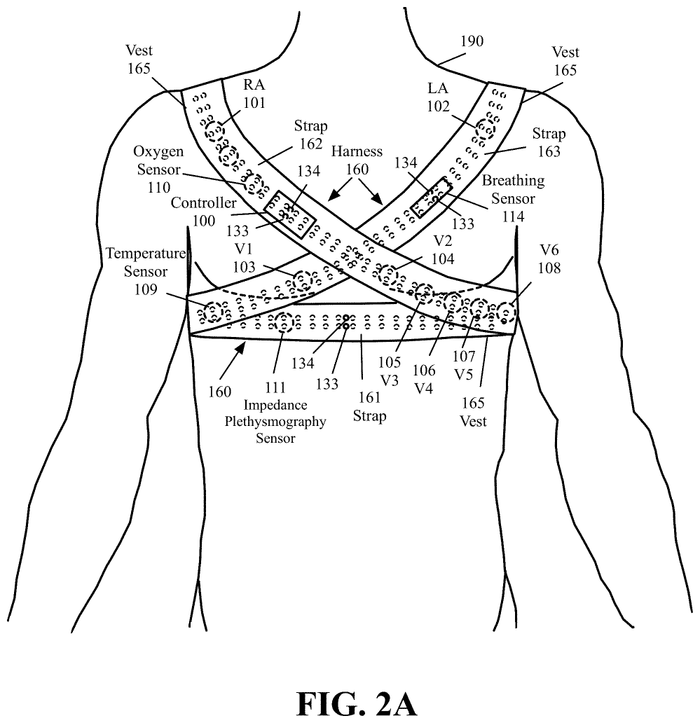

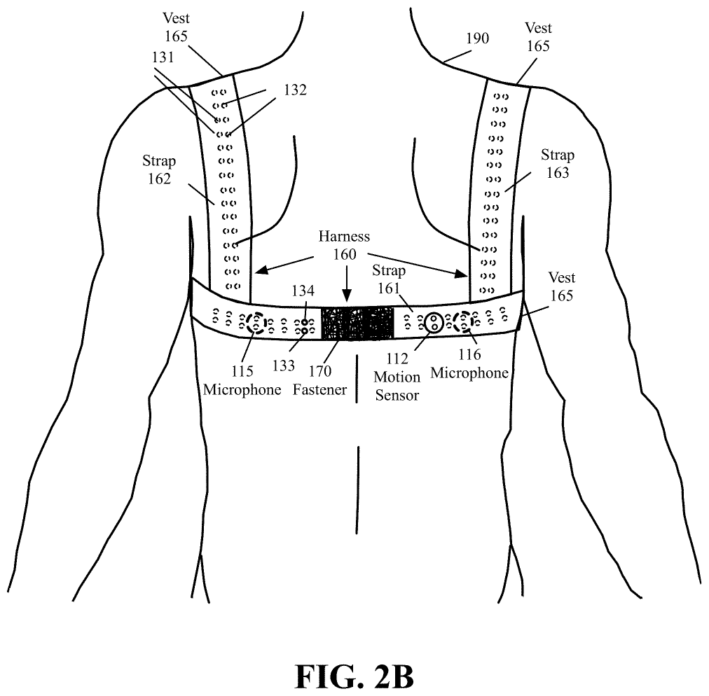

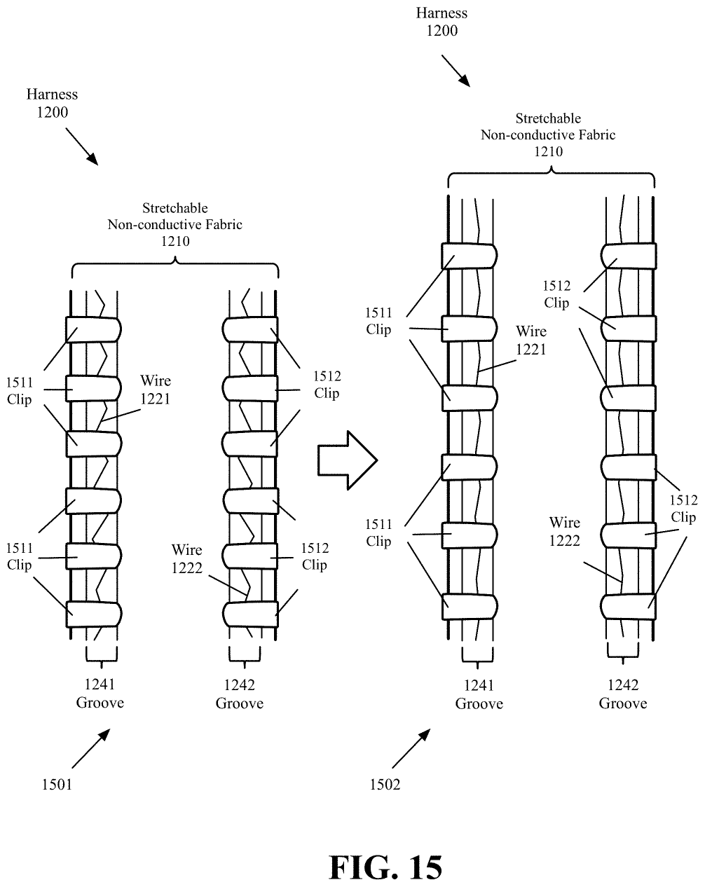

Although FIGS. 1A-1B and several other examples, such as FIGS. 2A-2B, 10A-10B, and 11A-11D, illustrate snap shape connectors, such as the connectors 131 and 132, for connecting sensor units and/or electronic devices to the bus of the wearable health monitoring harness, other embodiments may use other types of connectors such as, for example, and without limitations, clips or clamps for connecting sensor units and/or electronic devices to the bus. These embodiments are described below with reference to FIGS. 15-18 and 21-22.

FIG. 2A is a functional diagram illustrating a front view of an example embodiment of the wearable health monitoring harness 160 of FIGS. 1A-1B after attaching several sensor units to the wearable harness, according to various aspects of the present disclosure. FIG. 2B is the back view of the wearable health monitoring harness 160 of FIG. 2A, according to various aspects of the present disclosure.

With reference to FIG. 2A, a controller 100 and several sensor units 101-116 may be attached to the wearable harness 160. In the example of FIG. 2A, the sensor units may include ECG sensor units 101-108, a temperature sensor unit 109, an oxygen sensor unit 110, an impedance plethysmography sensor unit 111, a motion sensor unit 112, a breathing sensor unit 114, and microphone sensor units 115 and 116 (which may be used to receive sound waves from the person's lungs).

In the example of FIG. 2A, each sensor unit 101-111 and 115-116 may be connected to a pair of connectors 131-132 accessible from the bottom surface of the harness 160. The controller 100, the motion sensor unit 112, the breathing sensor unit 111, any display units, and/or any audio/video units may each be connected to a pair of connectors 133-134 accessible from top surface of the harness 160.

The ECG sensor units 101-108 may be used to generate an ECG, which is an electrical recording of the heart and may be used in monitoring and investigating of heart diseases. The temperature sensor unit 109, in some embodiments, may include a thermistor that may touch the body of the person 109 and may covert the temperature of the person into a voltage signal.

The oxygen sensor 110 may include sensor circuitry that measure a person's oxygen saturation. Some of the oxygen sensor units may operate in a transmissive pulse oximetry mode and may come into contact with a part of the body that has blood vessels close to the skin. Some of the oxygen sensor units may operate in a reflective pulse oximetry mode and may be placed, for example, and without limitations, on the chest of a person.

The impedance plethysmography sensor unit 111 may measure small changes in the electrical resistance of the chest or other regions of the body. These measurements may reflect blood volume changes. The impedance plethysmography sensor unit 111 may be used in conjunction with the ECG sensor units 101-108 to measure the cardiac output of the person 190. The motion sensor unit 112 may include one or more of a gyroscope, an accelerometer, a magnetometer, etc., to detect motion, which may be used, for example, and without limitations, to correct and/or to ignore portions of the ECG sensor units' signals that may be affected by the person's motion.

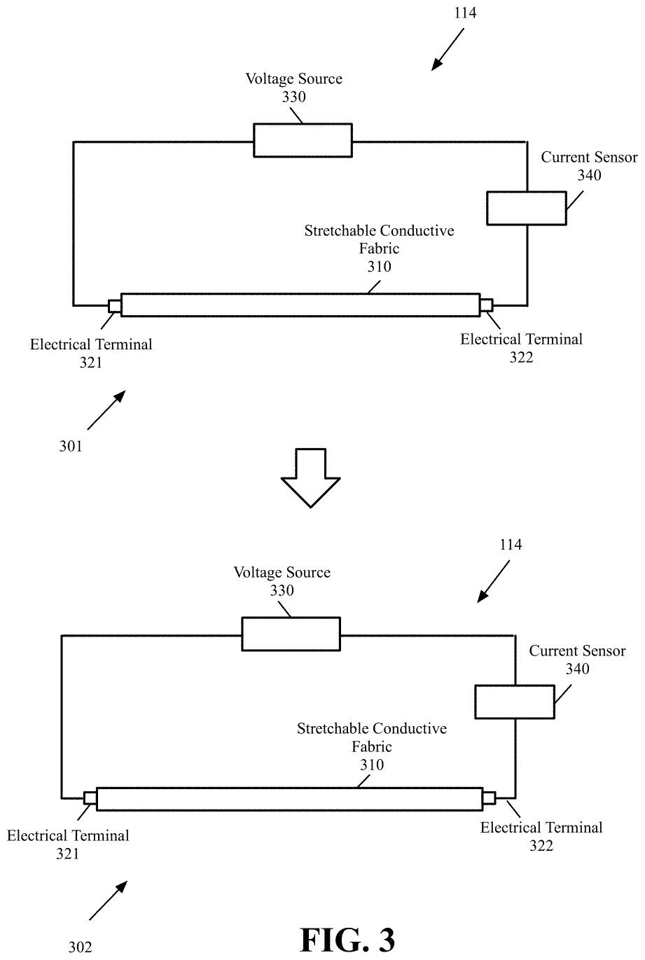

The breathing rate sensor unit 114 may measure the breathing rate of the person 190. FIG. 3 is a block diagram illustrating an example breathing rate sensor unit 114, according to various aspects of the present disclosure. With reference to FIG. 3, the breathing rate sensor unit 114 may include a stretchable conductive fabric 310. The stretchable conductive fabric 310 may conduct electricity and may change electrical resistance when stretched.

The stretchable conductive fabric 310 may be in the shape of a flexible cylindrical cord. The stretchable conductive fabric 310 used in the breathing rate sensor 114 unit of the different embodiments may be between 1 to 4 inches long and between 0.05-0.1 inch diameter when not stretched. The conductive fabric 310, in some embodiments, may be made with metal strands woven into the construction of the fabric or by metal-coated yarns. The conductive fabric 310, in some embodiments, may include a non-conductive substrate, which is coated or embedded with electrically conductive elements. One example of the stretchable conductive fabric is the conductive Lycra fabric.

With continued reference to FIG. 3, the stretchable conductive fabric 310 may be connected to the electrical terminals 321-322. The voltage source 330 may apply a constant voltage to the electrical terminals 321-322. The voltage source, in some embodiments, may be an active voltage source. In other embodiments, the voltage source 330 may receive power from the wearable health monitoring harness' battery (e.g., the battery 420 described below with reference to FIG. 4) and convert the received power to a fixed voltage.

FIG. 3, as shown, includes two operational stages 301-302. In stage 301, the breathing rate sensor unit 114 may be connected to a pair of connectors 131-132 (if connected to the bottom surface of the harness 160 of FIG. 2A) or to a pair of connectors 133-134 (if connected to the top surface of the harness 160). The stretchable conductive fabric 310, in stage 301, may not be stretched. In this stage, the stretchable conductive fabric 310 may have a resistance of R₁, which may be for example, and without limitations, in the order or 1000 ohms per linear inch. The current sensor 340, may therefore, measure a current of I₁=V/R₁, where V is the constant voltage applied by the voltage source 330.

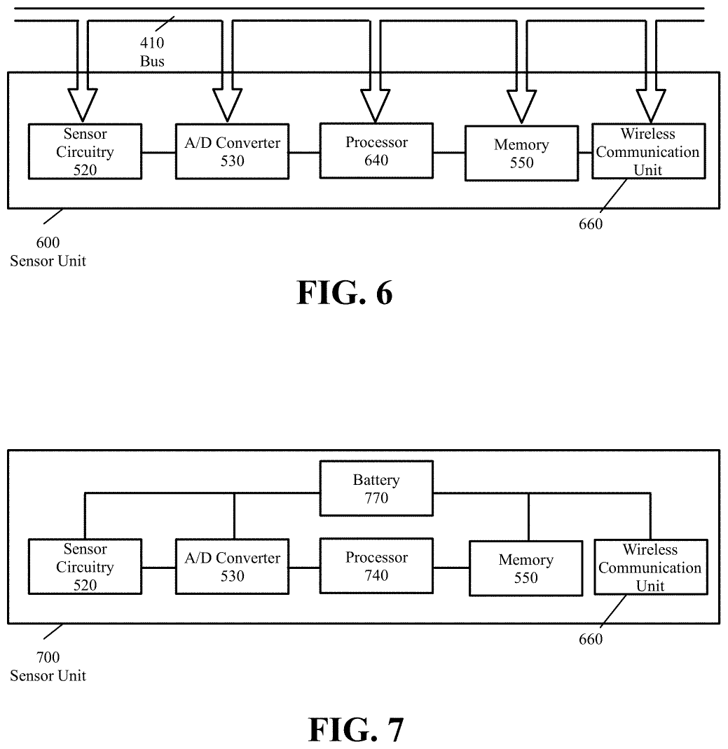

In stage 302, the stretchable conductive fabric 310 may be stretched due to the breathing of the person 190 who is wearing the wearable health monitor harness 160. When the stretchable conductive fabric 310 is stretched, the resistance of the stretchable conductive fabric 310 may increase to R₂>R₁. The current sensor 340 may measure the new current as I₂=V/R₂. Since the breathing rate sensor 114 is used to measure the breathing rate, the difference I₁-I₂ between the two current values I₁ and I₂ may be used as an indication of the stretchable conductive fabric 310 being stretched as the result of the person's breathing. As described below with reference to FIG. 4, the controller 100 of the harness 160 of the present embodiments may include a processor 440. As described below with reference to FIGS. 5-6, the sensor units of the present embodiments may include a processor 540. The processor of the controller 100 and/or the processor of 540 of the breathing rate sensor unit 114 may calculate the breathing rate of the person as the number of times that the current may change over a threshold in a given time interval.

The current sensor 340, in some embodiments, may be a Hall effect sensor that may be used to measure the current. It should be noted that, instead of a fixed voltage source 330 and a current sensor 340, some embodiments may use a fixed current source and a voltage sensor. These embodiments may measure the voltage changes due to the change of the resistance of the stretchable conductive fabric 310.

The breathing rate sensor 114 of FIG. 2A, in some embodiments, may be a pressure sensor, e.g., and without limitations, a piezoelectric sensor. The piezoelectric sensor may include a piezoelectric transducer that may generate a voltage in response to an applied force, pressure, or strain. In the embodiments that the breathing rate sensor 114 is a pressure sensor, the breathing rate sensor 114 may be connected to a pair of connector 131-132 that may be located on the bottom surface of the harness. As the person wearing the harness breaths, the pressure may generate a voltage in response to the pressure (or the force) caused by breathing. The controller 100 of the harness 160 may be configured to measure the breathing rate of the person who is wearing the harness by determining the number of times the difference between the minimum and the maximum voltages generated by the pressure sensor exceeds a threshold over a time period. The threshold may be determined by experiment and may be stored in a memory accessible by the controller of the harness 160.

Referring back to FIG. 2A-2B, the microphone sensor units 115 and 116 may include microphones that are configured to receive sound waves from the person's lungs. The microphone sensor units 115 and 116 are typically connected to the back of a person to receive sound waves from the bases of the person's lungs.

One of the technical advantages of the wearable health monitoring harnesses 160 of the present embodiments is the numerous connectors 131-134 that run across the length of the harness 160. None of the sensors unit 101-116 need to be installed on the harness 160 prior to the harness 160 being worn by the person 190. The position of each sensor unit 101-116 may be finetuned by connecting the sensor unit to one of the numerous pairs of connectors depending on the body size of different persons that may wear the harness 160.

For example, the person may wear the harness without any sensors unit attached. A medical professional may then connect each sensor to a pair of connectors such that the electrode of the sensor is placed on a correct location on the body. The medical professional may examine the signals generated by a sensor and may decide to move the sensor unit from the pair of connectors to which the sensor is currently connected to another pair of connectors and repeat this process unit the signal generated by the sensor unit meets an expected quality and strength. The wearable health monitoring harness of the present embodiments is configured with pairs of connectors that are positioned close to each other (e.g., within 1 inch, 2 inches, etc., of each other) in order to allow the proper positioning of the sensor units by moving the sensor units on different pairs of connectors on the harness until a position with proper signal quality and strength is found.

Another technical advantage of the wearable health monitoring harnesses of the present embodiments is replacing any defective or marginal sensor units without replacing the whole harness. Since the sensor units are pluggable, a defective sensor unit may be replaced by disconnecting the faulty sensor unit and connecting a new sensor unit. Medical establishments, such as, physician offices, hospitals, clinics, etc., may have spare sensor units and use them to quickly replace the faulty sensor units.

Another technical advantage of the wearable health monitoring harnesses of the present embodiments is the ease of upgrading the pluggable sensor units when new and/or different versions of sensors become available. For example, when a new temperature sensor unit, a new motion sensor unit, a new microphone sensor unit, etc., becomes available, the medical personnel may start using the new sensor units by plugging the new sensor units to a harness when a person wears the harness and the harness is being fitted with the sensor units.

FIG. 4 is a block diagram illustrating example components of a controller unit 100 of a wearable health monitoring harness, according to various aspects of the present disclosure. With reference to FIG. 4, the controller unit 100 may include a battery 420, a communication unit 430, a processor 440, and a memory unit 450. The battery 420, the communication unit 430, the processor 440, and the memory unit 450 may be connected to the bus 410 of the wearable harness. The controller unit 100, in some embodiments, may be on one or more printed circuit boards (PCBs). The controller unit 100, in some embodiments, may be a processor.

The electronic devices (e.g., the sensor units, the controller, the audio/video devices, etc., described above with reference to FIGS. 2A-2B) connected to the harnesses 160 may receive power and/or may communicate data with other electronic devices attached to the harness through the bus 410. In some embodiments, the bus 410 may include several wires that may run across the wearable health monitoring harness. For example, one wire may connect the connectors 131 and 133 (FIGS. 1A-2B). Another wire may connect the connectors 132 and 134. The wires may be used to provide electrical power from the battery 420 (FIG. 4) to the connectors 131-134 (FIGS. 1A-2B). The bus wires may be used to send and receive signals (e.g., commands, data, and/or status) between the electrical devices that are connected to the connectors 131-134.

The battery 420, in some embodiments, may be a rechargeable battery. Although FIG. 4 shows only one battery 420, some of the present embodiments may include several batteries 420. The battery (or batteries) 420 may provide electrical power to different components of the wearable health monitoring harness 160 (FIGS. 1A-2B).

With further reference to FIG. 4, the communication unit 430, in some embodiments, may include a radio transceiver (not shown) and one or more antennas (not shown). The radio transceiver may communicate over a communication link using, for example, and without limitations, one or more of the following protocols: Cellular (e.g., 2G, 3G, 4G, 5G, etc.), WLAN 802.11 (or Wi-Fi), Bluetooth®, Radio-Frequency Identification (RFID), Worldwide Interoperability for Microwave Access (WiMAX), HD Radio™, Ultra-wideband (UWB), ZigBee, 60 GHz, etc. In addition to, or in lieu of, the radio transceiver and the antenna, the communication unit 430, in some embodiments, may include a wired port for communicating through wires.

The memory 450 may be one or more units of similar or different memories. For example, the memory 450 may include, without any limitations, random access memory (RAM), read-only-memory (ROM), erasable programmable read-only memory (EPROM), electrically erasable programmable read-only memory (EEPROM), flash memory (e.g., secured digital (SD) cards, mini-SD cards, micro-SD cards, etc.), magnetic and/or solid state hard drives, and/or any other optical or magnetic storage media.

The processor 440 may be, for example, and without limitations, a microprocessor, a microcontroller, a digital signal processor (DSP), etc. The processor 440 may be, a single processor or a multi-core processor. The processor 440 may be configured to control the operation of one or more electronic devices that are attached to the wearable health monitoring harness 160. The processor 440 may be configured to communicate with one or more external electronic devices.

In some embodiments, the processor 440 may send signals over the bus 410 and may request different devices, such as the sensor units, audio/video devices, displays, etc., that are also connected to the bus, to send their identifications to the processor 440 over the bus 410. In other embodiments, each device that is connected to the bus 410 may broadcast its identification (e.g., once or at different intervals) over the bus 410. The identification of each device may identify the type and the function of the device to the processor 440.

The processor 440, in some embodiments, may control the intervals that other devices may use the bus 410 to send their digitized data to the processor 440. For example, the processor 440 may determine the communication interval based on the available bandwidth on the bus 410 and/or the number and the type of devices that are connected to the bus 410.

The processor 440, may perform different operations on the digitized data that the processor 440 receives from the electronic devices connected to the bus. For example, in the embodiments that use error detection and/or error correction codes for sending and transmitting data over the bus 410, the processor 440 may perform error detection and/or error correction on the digitized data received over the bus 410. The processor 440, may add error detection and/or error correction codes to the signals that the processor 440 sends to other electronic devices attached to the harness and/or external to the harness.

The processor 440 may perform data filtering to remove duplicate, redundant, and/or extraneous data. The processor 440, in some embodiments, may perform calculation algorithms on the data received from one or more sensor units, may compare sensor data with limits, may generate reports, error messages, alerts, etc. The processor 440, may store raw and/or processed data in the memory 450.

The processor 440, in some embodiments, may determine the blood pressure of the person who is wearing the harness using the ECG data received from the ECG sensor units 101-108. In some embodiments, the memory 450 may store an off-the-shelf or a custom made algorithm that may use the ECG data received from the ECG sensor units 101-108 and may estimate the blood pressure. The processor 440 may execute the algorithm to periodically estimate the blood pressure of the person wearing the harness and may store the values of the blood pressure in the memory 450. The processor 440, in some embodiments, may generate a warning signal and may send the warning signal to one or more external electronic devices if the blood pressure is not within a maximum and a minimum limit, which may depend on the age and health of the person wearing the harness.

The processor 440, in some embodiments, may perform health checking of the battery 420 (e.g., the charge level of the battery 420), the communication unit 430 (e.g., by sending and receiving communication packets through the communication unit), the processor 440 (e.g., performing self-health check), and/or the memory 450 (e.g., reading from and/or writing to specific memory locations). The processor 440, may store the health status of the individual components of the controller 100 and/or the overall health status of the controller 100 in the memory 450. The processor 440 may send the data stored in the memory 450 to external electronic devices through the communication unit 430. The processor 440 may display the data on display devices and/or on audio/video devices that are attached to the harness and/or are external to the harness.

In the embodiments that include a display on the wearable health monitoring harness 160, the processor 440 may display different data items and/or the health status of different electronic devices that are connected to the bus 410. In the embodiments that include an audio/video device on the wearable health monitoring harness 160, the processor 440 may record audio and/or video and my store the recorded audio and/or video in the memory 450 and/or may send the recorded audio and/or video to external devices using the communication unit 430.

It should be noted that the battery (or batteries) 420, in some embodiments, may not be on the same circuit board as the controller 100. In these embodiments, the battery (or batteries) 420 may be on one or more separate circuit boards that may be separately connected to the harness bus. In some embodiments, the processor 440 may perform some or all of the above-mentioned communication through a wireless link, in addition to, or in lieu of communicating through the bus 410.

FIG. 5 is a block diagram illustrating example components of a sensor unit 500 used with a wearable health monitoring harness, according to various aspects of the present disclosure. With reference to FIG. 5, the sensor unit 500 may include sensor circuitry 520, an analog to digital (A/D) converter 530, a processor 540, and a memory unit 550. The sensor circuitry 520, the A/D converter 530, the processor 540, and the memory unit 550 may be connected to the bus 410. The components of the sensor unit 500 may receive power from the battery (or batteries) 420 of FIG. 4 and/or may communicate data with the controller 100 over the bus 410.

The sensor unit 500 may include the sensor circuitry 520 that is configured to measure one or more bodily signals of a person. For example, and without limitations, the sensor circuitry 520 may be configured to measure temperature, oxygen level, breathing rate, pressure, ECG signals, sound from the lungs, blood pressure, motion, etc. The modular design of the wearable health monitoring harness of the present embodiments allows upgrading the sensor designs, replacing defecting sensors, replacing sensors with marginal performance, etc.

With further reference to FIG. 5, the A/D converter 530 may receive an analog signal from the sensor circuitry 520 and may convert the analog signal into a digital signal. The A/D converter, in some embodiments, may use the negative wire of the bus 410 as a reference voltage (e.g., and without limitations, as a 0 volt reference voltage). Converting the sensor data from analog to digital by the A/D converter 530 of the sensor unit 500, provides the technical advantage of preventing noise and interference when the data is sent from the sensor unit 500 to the controller 100. For example, in the prior art ECG monitors, each sensor is connected by a separate wire to the ECG monitor. The analog data of each sensor may be, for example, in the order of several millivolts. The weak analog signal has to travel over a long wire to the ECG monitor and may become subject to interference and noise.

On the other hand, the sensor units (e.g., the sensor unit 500 of FIG. 5, the sensor unit 600 of FIG. 6), the sensor unit 700 of FIG. 7, etc.) of the present embodiments provide the technical advantage of digitizing the analog data prior to sending the data to the controller 100. The processor 540 of the sensor units, in some embodiments, may add additional bits to the digitized data to provide error detection and/or error correction for additional integrity of the data that is sent from the sensor units 500 to the controller 100.

The wearable health monitoring harnesses of the present embodiments provide the technical advantage of minimizing the length and the number of wires that may be connected from the sensor units to the controller 100. The wearable health monitoring harnesses of the present embodiments provide the ability for the sensor units to collect the data at the source and at the best location on the body without the use of any lengthy leads and wires that may expose the raw signals to various external radio noise and interference.

The processor 540 may be, for example, and without limitations, a microprocessor, a microcontroller, a DSP, etc. The processor 540 may be, a single processor or a multi-core processor. The processor 540 may receive the digitized data from the A/D converter 530. The processor 540 may perform data processing and/or data filtering to remove duplicate, redundant, and/or extraneous data. The processor 540, in some embodiments, may perform health checking of the sensor circuitry 520, the A/D converter 530, the processor 540, and/or the memory 550. The processor 540, may store the health status of the individual components of the sensor unit 500 and/or the overall health status of the sensor unit 500 in the memory 550 for sending to the health status to the processor 440 of FIG. 4.

The processor 540, may store raw and/or processed data in the memory 550. The memory 550 may be one or more units of similar or different memories. For example, the memory 550 may include, without any limitations, RAM, ROM, EPROM, EEPROM, flash memory (e.g., SD cards, mini-SD cards, micro-SD cards, etc.), magnetic and/or solid state hard drives, and/or any other optical or magnetic storage media. The processor 540 may send the data stored in the memory 550 to the processor 440 of the controller 100 over the bus 410.

In some of the present embodiments, the sensor units may include wireless transducers and may send the sensor data to an electronic device (e.g., the controller 100) that may be attached to, or may be external to, the wearable health monitoring harness. FIG. 6 is a block diagram illustrating example components of a sensor unit 600 with a wireless communication unit used with a wearable health monitoring harness, according to various aspects of the present disclosure.

The sensor unit 600 may include sensor circuitry 520, an A/D converter 530, a processor 640, a memory unit 550, and a wireless communication unit 660. The sensor circuitry 520, the A/D converter 530, the processor 640, the memory unit 550, and the wireless communication unit 660 may be connected to the bus 410. The components of the sensor unit 600 may receive power from the battery 420 of FIG. 4 and/or may communicate data with the controller 100 over the bus 410.

The sensor circuitry 520, the A/D converter 530, and the memory 550 may be similar to the corresponding components of FIG. 5. The processor 640 may be, for example, and without limitations, a microprocessor, a microcontroller, a DSP, etc. The processor 640 may be, a single processor or a multi-core processor. The processor 640 may perform data processing and/or data filtering to remove duplicate, redundant, and/or extraneous data. The processor 640, may store raw and/or processed data in the memory 550.

The processor 640, in some embodiments, may perform health checking of the sensor circuitry 520, the A/D converter 530, the processor 640, the memory 550, and/or the wireless communication unit 660. The processor 640 may store the health status of the individual components of the sensor unit 600 and/or the overall health status of the sensor unit 600 in the memory 550 for sending to the health status to the processor 440 of FIG. 4.

The processor 640 may send the data stored in the memory 550 through the wireless communication unit 660 to an electronic device (e.g., the controller 100) that may be attached to, or may be external to, the harness. The wireless communication unit 660 may include a radio transceiver (not shown) and one or more antennas (not shown). The transceiver may, for example, and without limitations, communicate over a communication link using one or more of the following protocols: Cellular (e.g., 2G, 3G, 4G, 5G, etc.), WLAN 802.11, Bluetooth®, RFID, WiMAX, HD Radio™, UWB, ZigBee, 60 GHz, etc.

Some of the embodiments that use sensor units 600 with wireless communication units may not include a controller unit on the wearable health monitoring harness. Instead, the sensor units and other electronic devices that are attached to the wearable health monitoring harness (e.g., audio/video devices, displays, etc.) may receive control signals and may exchange data with a controller that is not attached to the wearable health monitoring harness.

FIG. 7 is a block diagram illustrating example components of a sensor unit, with a wireless communication unit and one or more batteries, used with a wearable health monitoring harness, according to various aspects of the present disclosure. The sensor unit 700 may include sensor circuitry 520, an A/D converter 530, a processor 740, a memory unit 550, a wireless communication unit 660, and one or more batteries 770. The sensor circuitry 520, the A/D converter 530, and the memory 550 may be similar to the corresponding components of FIGS. 5 and 6. The wireless communication unit 660 may be similar to the wireless communication unit 660 of FIG. 6.

With reference to FIG. 7, the sensor unit 700, in some embodiments, may not be connected to a bus. The sensor circuitry 520, the A/D converter 530, the processor 740, the memory unit 550, and the wireless communication unit 660 may receive power from one or more batteries 770 that may be included in, or attached to, the sensor unit 700. The battery (or batteries) 770, in some embodiments, may be rechargeable and/or replaceable.

The processor 740 may be, for example, and without limitations, a microprocessor, a microcontroller, a DSP, etc. The processor 740 may be, a single processor or a multi-core processor. The processor 740 may perform data processing and/or data filtering to remove duplicate, redundant, and/or extraneous data. The processor 740, may store raw and/or processed data in the memory 550.

The processor 740, in some embodiments, may perform health checking of the sensor circuitry 520, the A/D converter 530, the processor 740, the memory 550, the communication unit 660, and/or the battery 770 (e.g., the charge level of the battery). The processor 740, may store the health status of the individual components of the sensor unit 700 and/or the overall health status of the sensor unit 700 in the memory 550 for sending to the health status to the processor 440 of FIG. 4.

The processor 740 may send the data stored in the memory 550 to an external electronic device through the wireless communication unit 660. The embodiments that use sensor units 700 with wireless communication units may not include a controller unit on the wearable health monitoring harness. Instead, the sensor units and other electronic devices that are attached to the wearable health monitoring harness (e.g., audio/video devices, displays, etc.) may receive control signals and may exchange data with a controller that is not attached to the wearable health monitoring harness.

With reference to FIGS. 5-7, the sensor units of the present embodiments provide the technical advantage of placing each sensor circuitry 520 in a sensor unit (e.g., an integrated circuit (IC) chip) that is pluggable to connectors 131-134 at different locations on the wearable health monitoring harnesses of the present embodiments. Some of the sensor units 500, 600, 700 may include electrodes that may have to come into contact with a person's body. The connectors 131-134 on the harness allow the position of each sensor unit to be adjusted such that an ideal position for the sensor's electrode on the body may first be identified. The sensor unit may then be connected to a pair of connectors on the harness that are closest to the current position of the sensor unit. The sensor units 500, 600, and/or 700, in some embodiments, may be on one or more PCBs.

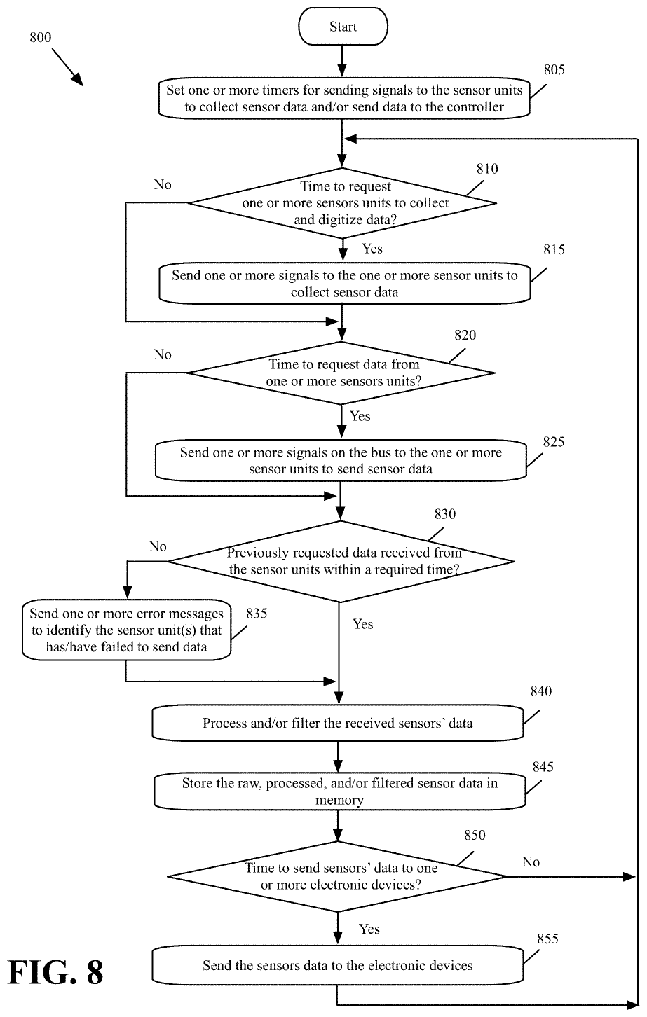

FIG. 8 is a flowchart illustrating an example process 800 for controller of a wearable health monitoring harness to collect and process the sensor units' data, according to various aspects of the present disclosure. The process 800, in some of the present embodiments, may be performed by the processor 440 of the controller 100 of FIG. 4.

With reference to FIG. 8, one or more timers may be set (at block 805) for sending signals to the sensor units to collect sensor data and/or send data to the controller. In some embodiments, the processor 440 (FIG. 4) may synchronize and/or control the operations of the electronic devices that are attached to the harness. In these embodiments, the processor 440 may periodically send signals (e.g., messages) to the electronic devices attached to the harness to collect and digitize sensor data, to send the digitized data, to display information (e.g., if the electronic device is a display unit or an audio/video device), etc.

For example, the processor 440 of FIG. 4 may send messages over the bus 410 to the sensor units 500 (FIG. 5) or 600 (FIG. 6) to collect sensor data. The processor 440 may send messages over a wireless link to the sensor units 600 (FIG. 6) or 700 (FIG. 7) to collect sensor data. In some embodiments, the controller may send the messages to one sensor unit at a time, to several related sensor units at a time (e.g., and without limitations to all sensor units that are collecting ECG related data), or to all sensor units at the same time. The processor 440 may maintain one or more timers to determine when to instruct the sensor units and/or other electronic devices that are connected to the harness to collect and digitize data and/or when to transmit the digitized data.

With further reference to FIG. 8, a determination may be made (at block 810) whether it is time to request one or more sensor units to collect and digitize data. For example, the processor 440 of FIG. 4 may periodically send messages to one or more sensor units to collect and digitize data. When a determination is made that it is not time to request one or more sensor units to collect and digitize data, the processing may proceed to block 820, which is described below.

Otherwise, one or more signals may be sent (at block 815) to the one or more sensor units to collect sensor data. For example, the processor 440 of FIG. 4 may send one or more messages to one or more sensor units 500 (FIG. 5), 600 (FIG. 6), 700 (FIG. 7), etc., to collect and digitize data. In some embodiments, each electronic device that is connected to the harness may include an identification. In these embodiments, the messages sent by the processor 440 to specific sensor units may identify the sensor units by the corresponding identifications. The identification may be used by the sensor units to access the messages that include their identification and ignore the messages that do not have their identification.

In some embodiments, the processor 440 may send the one or more signals to the sensor units 500 (FIG. 5) or 600 (FIG. 6) over the bus 410. In other embodiments where the sensor units include wireless communication capability, the processor 440 may send the one or more signals to the sensor units (700) over a wireless communication link.

At block 820, a determination may be made whether it is time to request data from one or more sensor units. For example, the processor 440 of FIG. 4 may periodically send messages to one or more sensor units to send the digitized data. When a determination is made that it is not time to request data from one or more sensor units, the processing may proceed to block 830, which is described below.

Otherwise, one or more signals may be sent (at block 825) to the one or more sensor units to send sensor data. For example, the processor 440 of FIG. 4 may periodically send messages to one or more sensor units requesting the sensor units to send the digitized data. The messages, in some embodiments, may include the identification(s) of the sensor unit(s).

At block 830, a determination may be made whether previously requested data is received from the sensor units within a required time. For example, the processor 440 may expect each sensor unit to send back the digitized sensor data within a time period after a request for data is sent (at block 825). The time period may be a predetermined value or may be set by the processor 440 based on the number of sensor units, the type of the senor units, and/or the communication bandwidth between the controller and the sensor units.

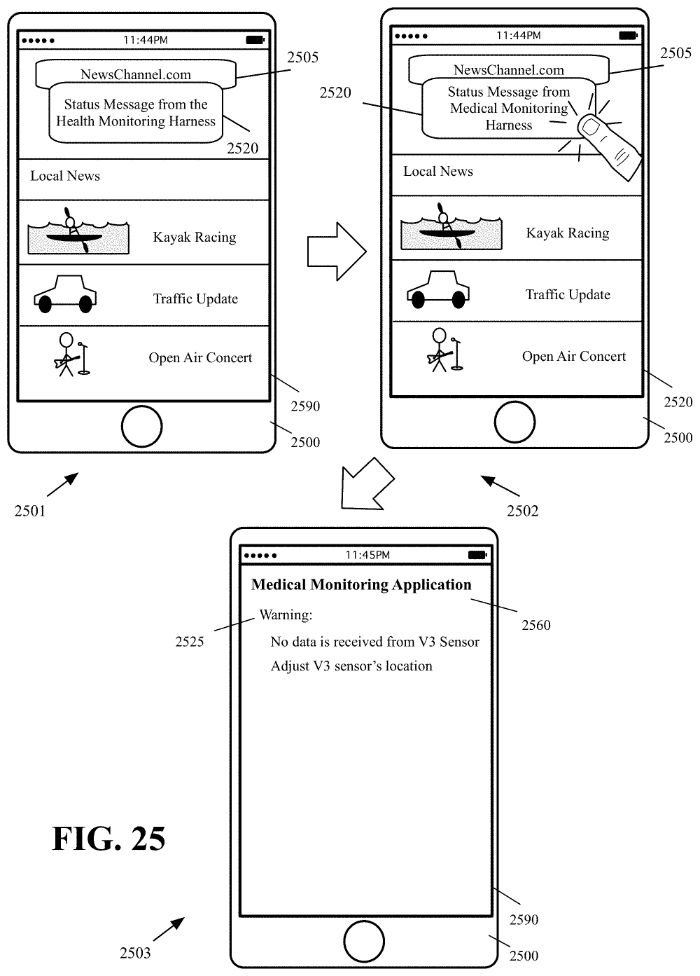

When a determination is made that previously requested data is received from the sensor units within the required time, the processing may proceed to block 840, which is described below. Otherwise, one or more error messages may be sent (at block 835) to identify the sensor unit(s) that has/have failed to send data. For example, as described below with reference to FIG. 25, the processor 440 may send one or more message to an external device to alert physicians, nurses, care providers, and/or the person who is wearing the harness, etc., to alert that one or more sensor units have failed to send their data. In addition to, or in lieu of sending the message(s) to the external device(s), the controller may send one or more messages to a display unit and/or to an audio/video device that is attached to the harness to identify the sensor unit(s) that failed to send data. The messages may allow adjusting and/or replacing the sensor unit(s) that is/are not sending data.

At block 840, the received sensors' data may be processed and/or filtered. For example, the processor 440, in some embodiments, may remove redundant sensor data, may perform error correction when error correction codes are used by the sensor units to communicate sensor data, and/or may remove erroneous data when error detection codes are used by the sensor units to communicate sensor data.

In some embodiments, the controller may perform calculation and/or algorithms to the sensor data. For example, instead of sending sensor data to an external electronic device to monitor ECG, the processor 440 may use sensor data from different sensor units to provide the leads required for ECG monitoring. The processor may then display the ECG curves on a display attached to the harness and/or may send the data to an external device such as, for example, and without limitations a mobile device to display the ECG curves.

The raw, processed, and/or filtered data may be stored (at block 845) in memory. For example, the processor 440 of FIG. 4 may store the sensor data in the memory 450, which may be volatile and/or non-volatile. A determination may be made (at block 850) whether it is time to send the sensors' data to one or more electronic devices. For example, the processor 440, in some embodiments, may send the sensors data to one or more external electronic devices, such as, for example, and without limitations, to hospitals', clinics' and/or care providers' computers, to one or more mobile devices associated with physicians, nurses, care providers, and/or the person who is wearing the harness. In some embodiments, the processor 440 may send sensors' data (e.g., processed sensor data) to electronic devices such as, for example and without limitations, display units and/or audio/video devices attached to the harness.

When it is determined that it is not time to send the sensors' data to one or more electronic devices, the processing may proceed back to block 810, which was described above. Otherwise, the sensors data may be sent to the electronic devices. The processing may then proceed to block 810, which was described above.

The specific operations of the process 800 may not be performed in the exact order shown and described. Furthermore, the specific operations described with reference to FIG. 8 may not be performed in one continuous series of operations, in some aspects of the present disclosure, and different specific operations may be performed in different embodiments.

For instance, in some aspects of the present embodiments, the order of operations 810, 820, 830, and/or 850 may be performed in a different order. In some embodiments, the processor 440, may send (at block 815) one or more signals to one sensor unit, to a group of two or more sensor units, or all sensor units to collect sensor data. In some embodiments, the processor 440, may send (at block 825) one or more signals to one sensor unit, to a group of two or more sensor units, or all sensor units to send sensor data.

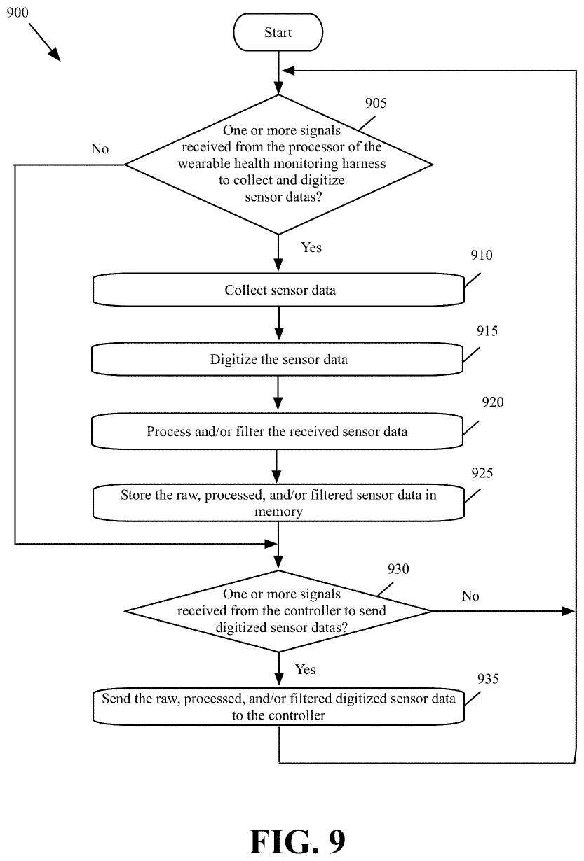

FIG. 9 is a flowchart illustrating an example process 900 for sensor unit of a wearable health monitoring harness to collect, process, and/or send data, according to various aspects of the present disclosure. The process 900, in some of the present embodiments, may be performed by the processor 540 (FIG. 5), 640 (FIG. 6), and/or 740 (FIG. 7) of a sensor unit.

With reference to FIG. 9, a determination may be made whether one or more signals are received (at block 905) from the processor 440 of the wearable health monitoring harness to collect and digitize sensor data. For example, the sensor units 500, 600, and/or 700 may receive one or more signals from the processor 440, as described above with reference to FIG. 8 to collect and digitize sensor data. When a determination is made that one or more signals are not received from the processor 440 to collect sensor data, the processing may proceed to block 930, which is described below.

Otherwise, sensor data may be collected (at 910). The sensor data may then be digitized (at block 915). For example, the sensor unit 500, 600, or 700, may digitize the analog data that the sensor circuitry 520 has received from a person's body.

The sensor's data may then be processed and/or filtered (at block 925). For example, the processor 540 (FIG. 5), 640 (FIG. 6), and/or 740 (FIG. 7) of a sensor unit may add error detection and/or error correction codes to the digitized data. The processor of the sensor unit may smooth the data, may filter noise, etc. The raw, processed, and/or filtered data may then be stored in memory. For example, the processor of the sensor unit may store the raw, processed, and/or filtered data in the memory 550, which may be volatile and/or non-volatile memory.

At block 930, a determination may be made whether one or more signals are received from the processor 440 of the wearable health monitoring harness to send digitized sensor data. For example, the processor 440 of FIG. 4 may send one or more signals to the sensor unit to request the sensor unit to send digitized sensor data, as described above with reference to FIG. 8. When a determination is made that signal(s) are not received from the controller to send digitized sensor data, the processing may proceed to block 905, which was described above.

Otherwise, the raw, processed, and/or filtered digitized data may be sent (at block 935) to the processor 440 of the wearable health monitoring harness. The processing may then proceed to block 905, which was described above.

The specific operations of the process 900 may not be performed in the exact order shown and described. Furthermore, the specific operations described with reference to FIG. 9 may not be performed in one continuous series of operations, in some aspects of the present disclosure, and different specific operations may be performed in different embodiments. For instance, in some embodiments, sensor unit may constantly receive analog data from the person's body. In these embodiments, operation 910 may not be done only in response to receiving one or more signals from the processor 440.

With reference to FIGS. 8 and 9, one technical advantage of the wearable health monitoring harness and the sensor units of the present embodiments is the ability of operating the sensor units independently but in synch with the instructions from the processor 440 of (FIG. 4) of the controller 100. The sensor units may collect analog sensor data, may digitize the analog data, and may store the digitized data in the sensor unit's local memory. The sensor units may then send the digitized data to the processor 440 per instructions received from the processor 440.

One exemplary example of a wearable health monitoring harness of the present embodiment was described above with reference to FIGS. 1A-2B. The wearable health monitoring harness, in other embodiments, may have different shapes and different configurations, such as, for example, and without limitations, different number shapes of straps, different number of straps, different arrangements of the straps, zero or more back sections, etc. Several other examples of wearable health monitoring harness of the present embodiment are provided below.

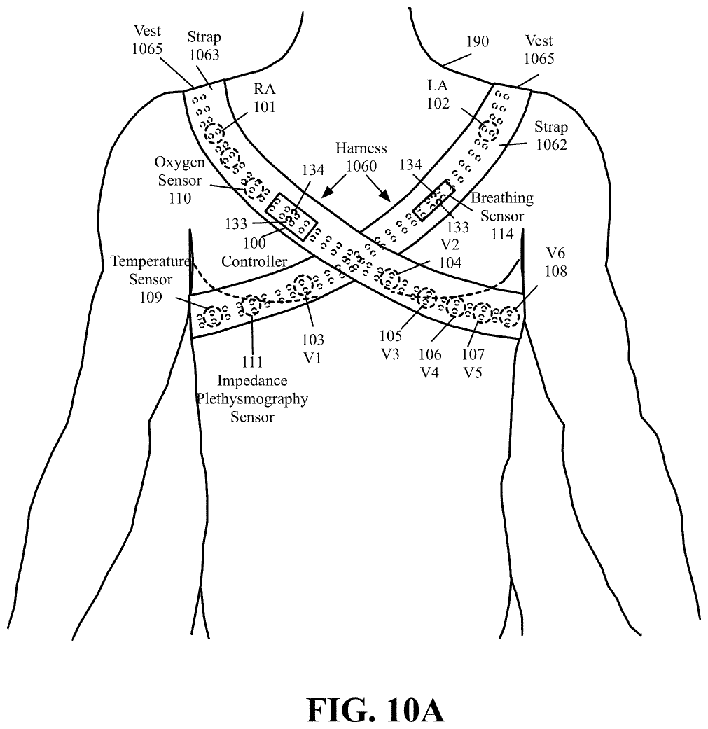

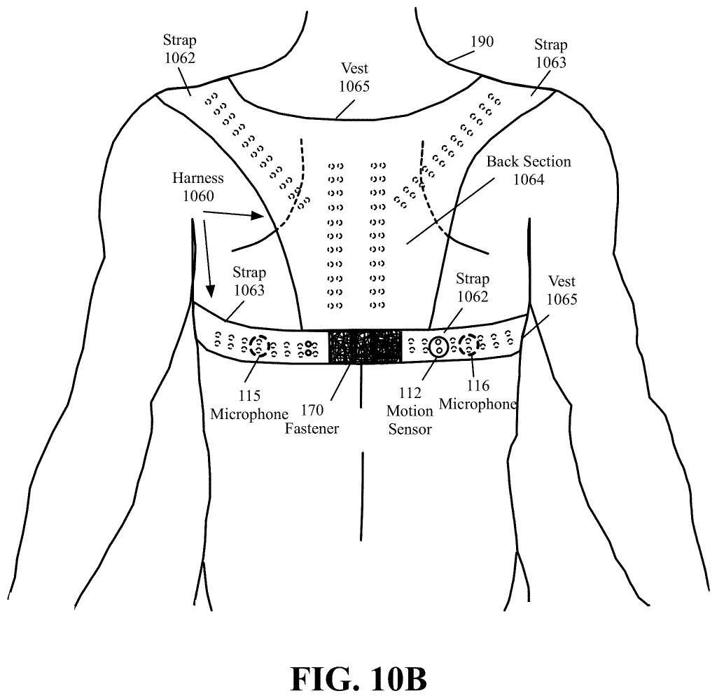

FIG. 10A is a functional diagram illustrating a front view of an alternative example embodiment of a wearable health monitoring harness 1060, after attaching several sensor units to the wearable harness, according to various aspects of the present disclosure. FIG. 10B is the back view of the wearable health monitoring harness 1060 of FIG. 10A, according to various aspects of the present disclosure.

With reference to FIGS. 10A and 10B, the wearable health monitoring harness 1060 may include the vest 1065. The vest 1065 may include the straps 1062 and 1063. The vest 1065 may include a back section 1064 that may be attached to the two straps 1062 and 1063. The straps 1062 and 1063, after being worn by the person 190, may go over the person's shoulder and may cross each other over the chest area. The straps 1062 and 1063 may go around the lower chest area and may be join to each other with a fastener 170. The back section 1064 may cover the upper back of the person wearing the harness.

The fastener 170 may be, for example, and without limitations, a hook-and-loop fastener, a set of buttons, etc., to allow for the harness 1060 to be quickly put on or off a person's body. The back section 1064 and the straps 1062 and 1063 may be made of a single piece of material or may be separate pieces that may be connected by fasteners, by sewing, by buttons, by stitches, etc.

Similar to the wearable health monitoring harness 160 of FIGS. 1A-2B, the wearable health monitoring harness 1060 of FIG. 10 may include the connectors 131-134 that may be connected to a bus that connects the connectors to one or more batteries and/or to a controller that is located on the harness 160.

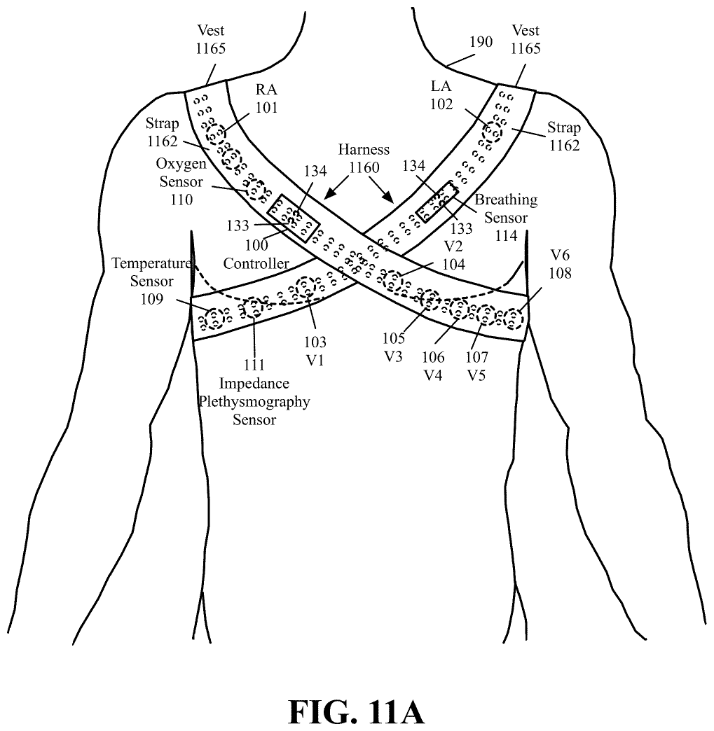

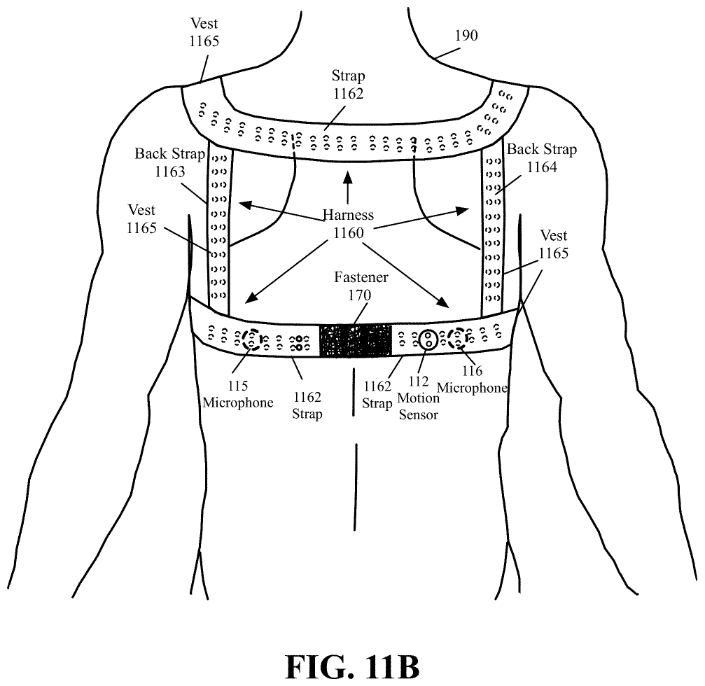

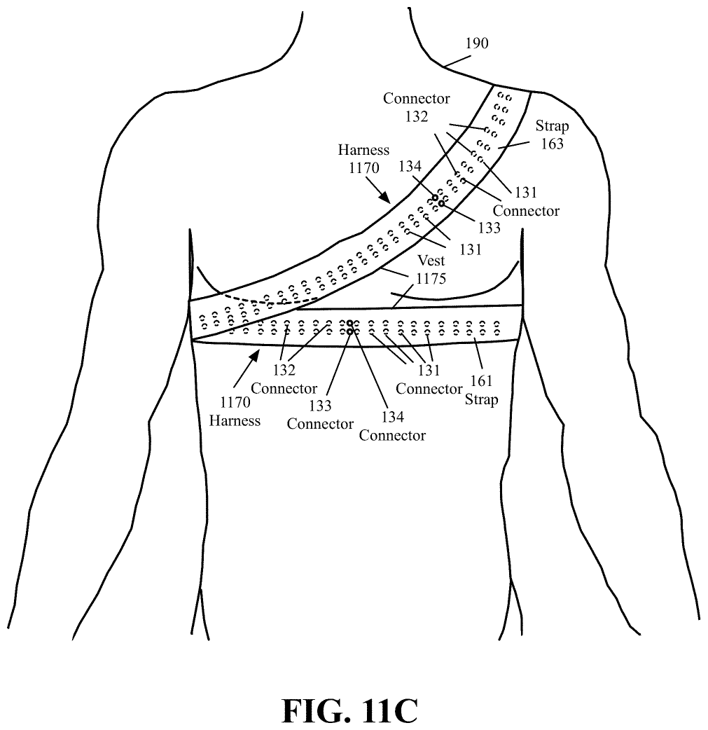

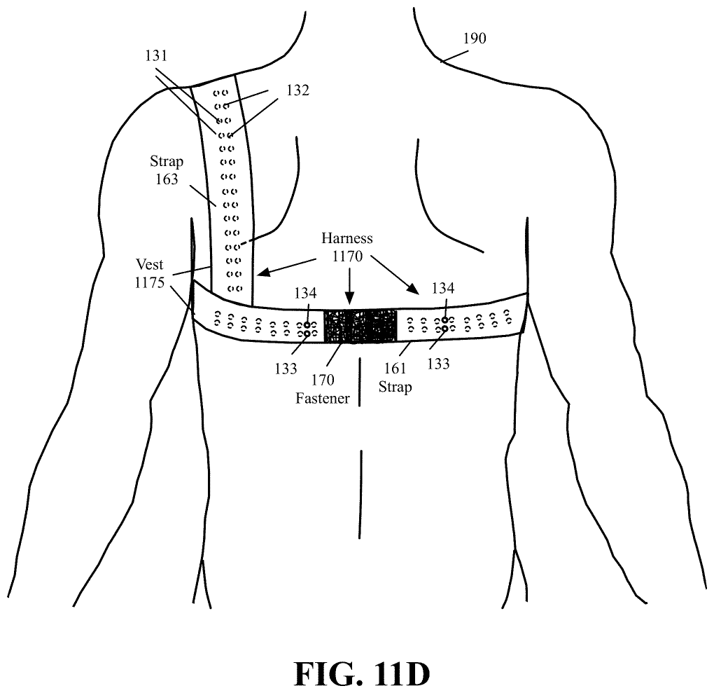

FIG. 11A is a functional diagram illustrating a front view of an alternative example embodiment of a wearable health monitoring harness 1160, after attaching several sensor units to the wearable harness, according to various aspects of the present disclosure. FIG. 11B is the back view of the wearable health monitoring harness 1160 of FIG. 11A, according to various aspects of the present disclosure.

With reference to FIGS. 11A and 11B, the wearable health monitoring harness 1160 may include the vest 1165. The vest 1165 may include the straps 1162, 1163, and 1164. The strap 1162 may form an arc section (FIG. 11B) on the upper back and shoulders of the person 190, may form an X shape in front of the chest (FIG. 11A) and go around the torso in the back (FIG. 11B). With reference to the strap 1162, the arc section and the section that goes around the torso in the back may be connected with two substantially parallel straps 1163 and 1164.

The straps 1163 and 1164 may be connected to the strap 1162 by fasteners, by sewing, by buttons, by stitches, etc. The strap 1162, in some embodiments, may include (as shown in FIG. 11B) a fastener 170, such as, for example, and without limitations, a hook-and-loop fastener, a set of buttons, etc., to allow for the harness 1160 to be quickly put on or off a person's body. Although in the depicted embodiment, the fastener 170 is on the back portion of the strap 1160, the fastener 170, in other embodiments, may be placed on the front portion of the strap 1162.

Similar to the wearable health monitoring harness 160 of FIGS. 1A-2B, the wearable health monitoring harness 1160 of FIGS. 11A-11B may include the connectors 131-134 that may be connected to a bus that connects the connectors to one or more batteries and/or to a controller that is located on the harness 160.

FIG. 11C is a functional diagram illustrating a front view of an alternative example embodiment of a wearable health monitoring harness 1170, according to various aspects of the present disclosure. FIG. 11D is the back view of the wearable health monitoring harness 1170 of FIG. 11C, according to various aspects of the present disclosure. With reference to FIGS. 11C and 11D, the wearable health monitoring harness 1170 may be similar to the wearable health monitoring harness 160 of FIGS. 1A-1D, except the vest 1175 of the wearable health monitoring harness 1170 of FIGS. 11C-11D does not include the strap 162.

As shown in FIGS. 11C-11D, the strap 161 may be worn around the chest, for example below the pectoral line. The strap 163 may be worn from the left shoulder of the person 190, going diagonally from the left shoulder around the upper body. The strap 161 may be connected to the strap 163 in front and in the back of the harness 160. The strap 161 may be connected to the strap 163 by fasteners, by sewing, be snap connectors, by buttons, by stitches, etc. The fasteners, in some embodiments, may be hook-and-loop fasteners.

With reference to FIG. 1D, the strap 161, in some embodiments, may include a fastener 170, such as, for example, and without limitations, a hook-and-loop fastener, a set of buttons, a set of snap connectors, etc., to allow for the harness 160 to be quickly put on or off a person's body. Although in the depicted embodiment the fastener 170 is on the back portion of the strap 161, the fastener 170, in other embodiments, may be placed on the front portion of the strap 161.

Similar to the wearable health monitoring harness 160 of FIGS. 1A-2B, the wearable health monitoring harness 1170 of FIGS. 11C-11D may include the connectors 131-134 that may be connected to a bus that connects the connectors to one or more batteries and/or to a controller that is located on the harness 1170. The wearable health monitoring harness, in some embodiments, may be a mirror (or reverse) of the wearable health monitoring harness of FIGS. 11C-11D. In these embodiments, the strap 163 may be worn diagonally from the right shoulder of the person 190.

In some embodiments, the bus 410 (FIGS. 4-6) may include several wires that may run across the wearable health monitoring harness. The vest of the wearable health monitoring harness, in some embodiments, may be made of a stretchable fabric in order to be comfortably worn by a person. The stretchable fabric may provide a tensile force with the body of a person to maintain reliable contact between the sensor unit's electrodes and the person's body. The stretchable fabric may also provide comfort when the wearable health monitoring harnesses are worn by different persons with different sizes.

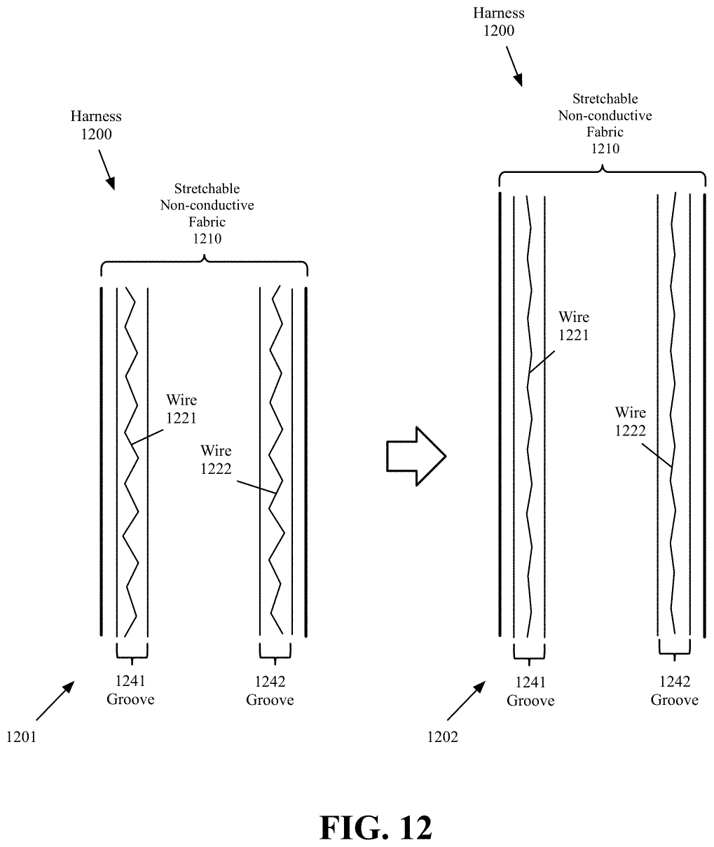

In order to prevent damage to the bus wires when the vest's fabric is stretched, some embodiments may provide slack in the bus wires when the fabric is not stretched. FIG. 12 is a top view of a portion of an example wearable health monitoring harness 1200 with a two-wire bus running across the harness, according to various aspects of the present disclosure. With reference to FIG. 12, the wearable health monitoring harness 1200 may, at least partially, be made of a non-conductive stretchable fabric 1210. The stretchable fabric 1210 may include the grooves 1241-1242 made from the same stretchable material as the stretchable fabric 1210. The wires 1201-1202 may run across the length of the grooves 1241-1242, respectively.

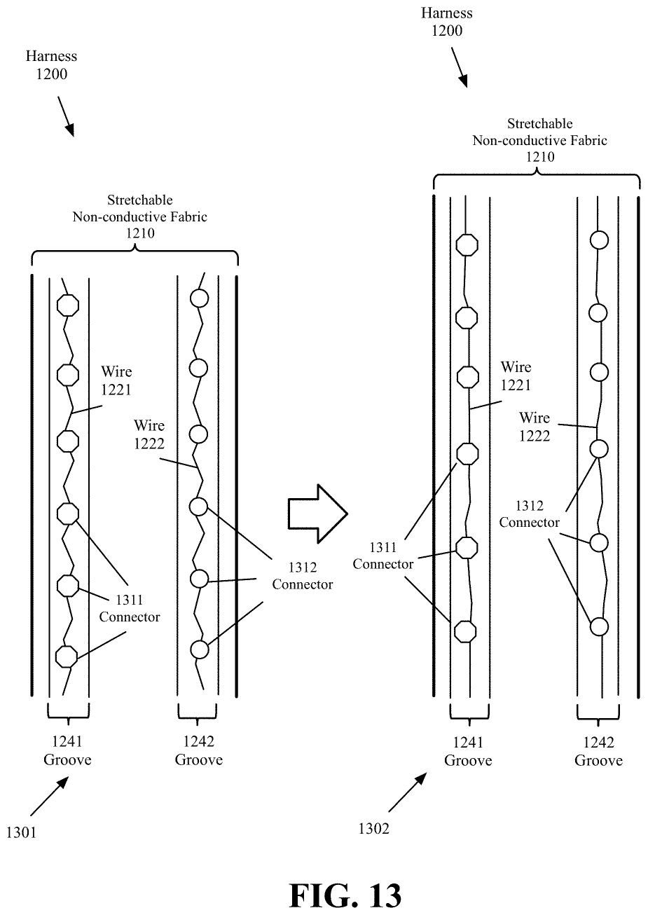

Different embodiments may provide different types of connectors, such as snap connectors, clips, clamps, etc., for connecting electronic devices, such as the controller, the sensor units, displays, audio/video devices, etc., to the bus. These embodiments are described below with reference to FIGS. 13-18. For clarity, the connectors are not shown in FIG. 12 in order to illustrate how the wires 1221-1222 may be stretched when the fabric 1210 is stretched.

FIG. 12, as shown, includes two operational stages 1201 and 1202. In stage 1201, the fabric 1210 is not stretched. As shown, the wires 1221-1222 may be embedded through the fabric 1210 in a sawtooth shape or in a wavy shape. In several examples shown herein, the wires 1221-1222 are shown in a sawtooth (or crisscross) shape. In should be understood that the wires 1221-1222 may be embedded through the fabric 1210 in a wavy shape or a combination of wavy and sawtooth shapes.

In stage 1202, the fabric 1210 may be stretched, for example, when the wearable health monitoring harness 1200 is worn by a person or is otherwise stretched. In stage 1202, as the fabric 1210 and the grooves 1241-1242 are stretched, the wires 1221-1222 may also be stretched, preventing damage and stress to the wires 1221-1222.

The wearable health monitoring harness, in some embodiments, may include connectors (e.g., the connectors 131-134 of FIGS. 1A-2B) for connecting electronic devices such as the controller, the sensor units, displays, audio/video devices, etc., to the bus. FIG. 13 is top view of a portion of an example wearable health monitoring harness with a two-wire bus and a set of snap connectors for connecting electronic devices to the bus, according to various aspects of the present disclosure.

With reference to FIG. 13, the stretchable fabric 1210, the grooves 1241-1242, and the wires 1221-1222 may be similar to the corresponding components of FIG. 12. FIG. 13 further illustrates a set of connectors 1311-1312 that are connected to the wires 1221-1222, respectively. The connectors 1311 may, for example, be similar to the connectors 131 or 133 of FIGS. 1A-2B. The connectors 1312 may, for example, be similar to the connectors 132 or 134 of FIGS. 1A-2B. The shape of the connectors 1311 and 1312 may be different (e.g., and without limitations, one of the connectors may be in the shape of a polygon and the other connector may be in the shape of a circle) in order to distinguish between the connectors 1311 and 1312, which are attached to different wires 1221-1222.

Some of the connectors 1311-1312 may be connected to the corresponding wires such the connectors may be accessible from the bottom surface of the harness 1200 (e.g., to be accessible from the surface of the harness that touches the body of a person wearing the harness). For example, some of the connectors 1311-1312 may be similar to the connectors 131 and 132 of FIGS. 1A-2B, respectively. Some of the connectors 1311-1312 may be connected to the corresponding wires such the connectors may be accessible from the top surface of the harness 1200 (e.g., to be accessible from the surface of the harness that is opposite to the body of the person wearing the harness). For example, some of the connectors 1311-1312 may be similar to the connectors 133 and 134 of FIGS. 1A-2B, respectively.

The connectors 1311-1312, in some embodiments, may be snap connectors that may mate with a corresponding snap connector port on the senor units, on the controller, and/or on other electronic devices that may be attached to the wearable health monitoring harnesses of the present embodiments. The connectors 1311-1312, in some embodiments, may be male and/or female pins that may mate with a corresponding female/male connector port on the senor units, on the controller unit, and/or on other electronic devices that may be attached to the wearable health monitoring harnesses of the present embodiments.

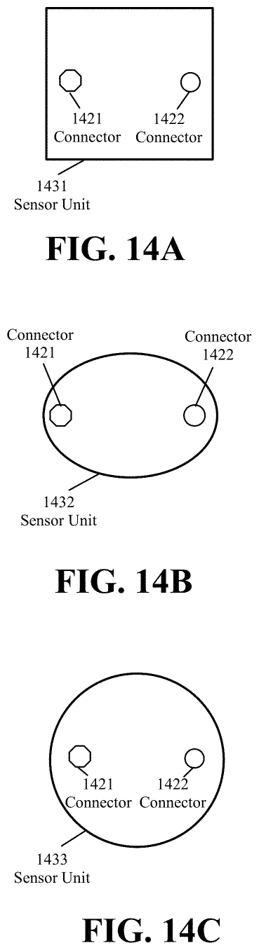

FIGS. 14A-14C illustrate example electronic devices 1431-1433 that may be connected to the connectors on the bus of an example wearable health monitoring harness, according to various aspects of the present disclosure. FIGS. 14A-14C may show the top views of the electronic devices, such as the ECG sensor units, the body temperature sensor units, the oxygen sensor units, the breathing rate sensor units, the impedance plethysmography sensor units, the microphone sensor units (e.g., for recording lungs' sounds), etc., that may be connected to the bottom surface of the wearable health monitoring harness to read the physiological and the vital signs of a person.

FIGS. 14A-14C may also show the bottom views of the electronic devices, such as the breathing rate sensors, the motion sensors, the controller, the batteries, the audio/video devices, etc., that may be connected to the top surface of the wearable health monitoring harness. The electronic devices 1431-1433 may include the connector ports 1421-1422 that may snap into a pair of corresponding connectors 1311-1312 of FIG. 13. The electronic devices 1431-1433 may have different shapes depending on the type of the sensor unit. Other details of the electronic devices 14A-14C are not shown for simplicity.

Referring back to FIG. 13, the figure includes two operational stages 1301 and 1302. In stage 1301, the fabric 1210 may not be stretched. In stage 1302, the fabric 1210 may be stretched, for example, when the wearable health monitoring harness 1200 is worn by a person or is otherwise stretched. In stage 1302, as the fabric 1210 and the grooves 1241-1242 are stretched, the wires 1221-1222 may also be stretched, preventing damage and stress to the wires 1221-1222.

The wearable health monitoring harness 1200 may include flaps (not shown) to cover the grooves after the wires are installed in the grooves. The flaps may be made of the stretchable material as the stretchable fabric 1210. One side of each flap may be attached to one side of a groove. The flap may fit snuggly inside the groove to prevent the wire to come out of the groove and/or to prevent damage to the wire. A portion of the flap may be pushed aside to expose a pair of connectors (e.g., a pair of connectors 1311-1312 of FIG. 13) in the groove in order to connect an electronic device, such as a sensor unit, or the controller, to the pair of connectors.

The wearable health monitoring harness, in some embodiments, may include clips and/or clamps for connecting electronic devices such as the controller, the sensor units, displays, audio/video devices, etc., to the bus. FIG. 15 is top view of a portion of an example wearable health monitoring harness with a two-wire bus and a set of clips for connecting electronic devices to the bus, according to various aspects of the present disclosure.

With reference to FIG. 15, the stretchable fabric 1210, the grooves 1241-1242, and the wires 1221-1222 may be similar to the corresponding components of FIGS. 12 and 13. FIG. 15 further illustrates a set of clips 1511-1512 that are connected to the wires 1221-1222, respectively. The clips 1511 may, for example, function similar to the connectors 131 or 133 of FIGS. 1A-2B. The clips 1512 may, for example, function similar to the connectors 132 or 134 of FIGS. 1A-2B. Although several examples are described herein with reference to clips, some embodiments may use clamps to achieve similar functionalities as the clips.

The sensors, in some embodiments, may only be a few millimeters thick (e.g., comparable to the thickness of a credit card) and a may have electrically conductive connector ports on their edges. The clips, in some embodiments, may be spring loaded or may otherwise be able to grip the electrically conductive connector ports of the sensors. At least the portions of the clips or clamps that come in contact with the bus and with the electrically conductive connector ports of the sensors may be electrically conductive.

In some embodiments, electronic devices such as batteries, display units, audio/video devices that may be thicker than a few millimeters may include, at least around their edges, thin base plates (e.g., a few millimeters thick) with electrically conductive connector ports that may be attached to the clips in similar manner as the sensors.

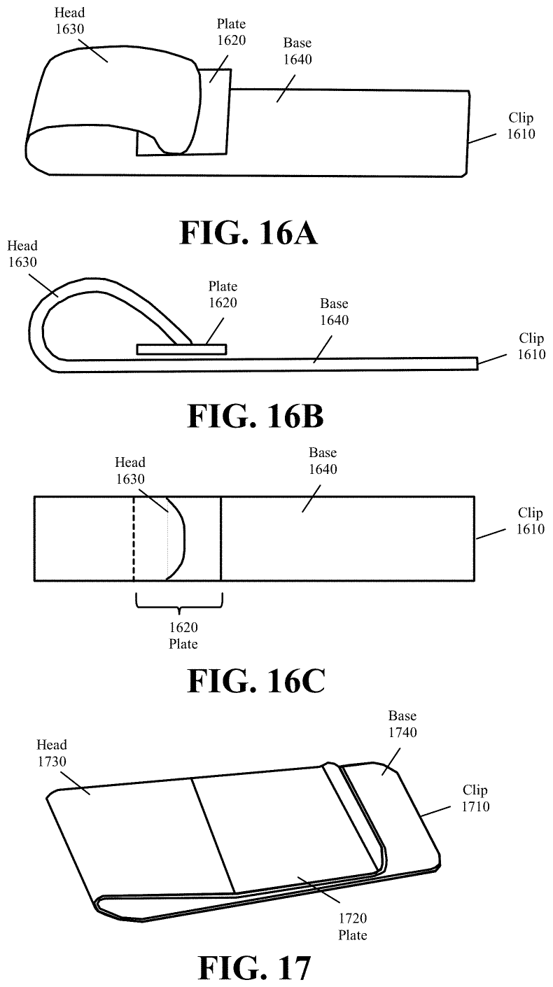

FIG. 16A is a top perspective view of an example clip for connecting the bus of a wearable health monitoring harness to sensors and/or electronic devices that may be attached to the wearable health monitoring harness, according to various aspects of the present disclosure. FIG. 16B is a side elevation view and FIG. 16C is a top view of the clip of FIG. 16A, according to various aspects of the present disclosure.

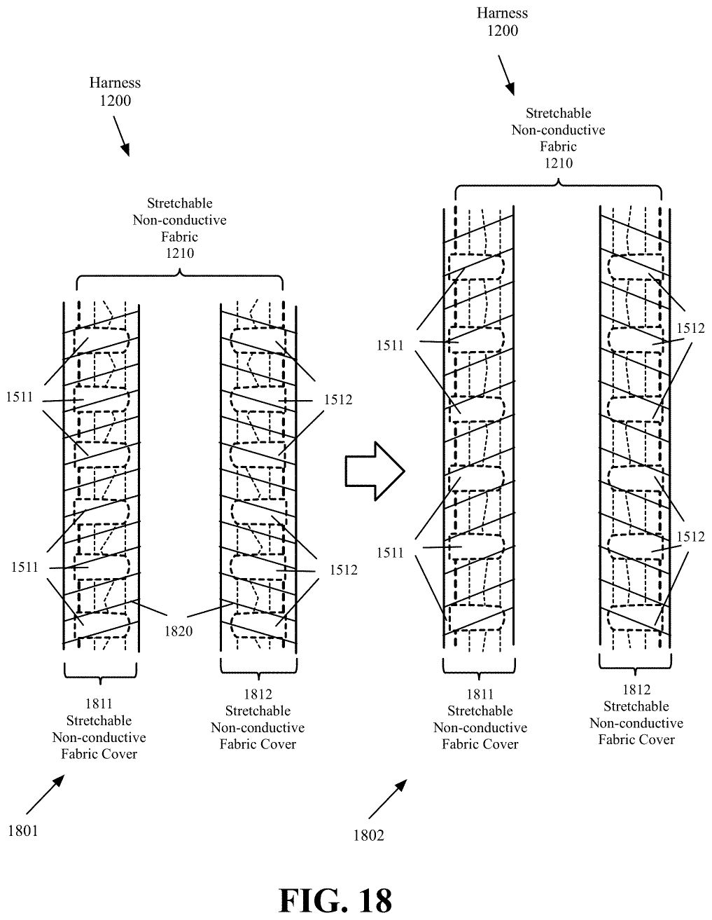

With reference to FIGS. 16A-16C, the clip 1610 may include a base 1640 and a head 1630. A plate 1620 may be attached to the head 1630. The head 1630 may be configured to act as a spring to pressure the plate 1620 against the base 1640 to make contact with a sensor and/or an electronic device. At least the portion of the plate 1620 that may come in contact with a sensor or electronic device may be made of conductive material. The base 1640 and/or the head 1630, in some embodiments, may be made of a non-conductive material such as, for example, and without limitations, plastic. In other embodiments, the base 1640 and/or the head 1630 may be made of conductive material and may be covered with non-conductive material, as described below with reference to FIG. 18.

In order to connect a sensor to the clip 1610, the head 1630 and the plate 1620 may be separated from the base 1640 by force and the edge of the sensor may be inserted in the gap between the plate 1620 and the base 1640. Once the force is removed from the head 1630, the head 1630 and the plate 1620 may tightly connect to the electrically conductive connector ports of the sensor. An electrically conductive connector port of an electronic device may also be attached to the clip in similar manner.

FIG. 17 is a top perspective view of another example clip for connecting the bus of a wearable health monitoring harness to sensors and/or electronic devices that may be attached to the wearable health monitoring harness, according to various aspects of the present disclosure. With reference to FIG. 17, the clip 1710 may include a base 1740 and a head 1730. A plate 1720 may be attached to the head 1730. The head 1730 may be configured to act as a spring to pressure the plate 1720 against the base 1740. At least the portion of the plate 1720 that may come in contact with a sensor or electronic device may be made of conductive material. The base 1740 and/or the head 1730, in some embodiments, may be made of a non-conductive material such as, for example, and without limitations, plastic. In other embodiments, the base 1740 and/or the head 1730 may be made of conductive material and may be covered with non-conductive material, as described below with reference to FIG. 18.

In order to connect a sensor to the clip, the head 1730 and the plate 1720 may be separated from the base 1740 by force and the edge of the sensor may be inserted in the gap between the plate 1720 and the base 1740. Once the force is removed from the head 1630, the head 1730 and the plate 1720 may tightly connect to the electrically conductive connector ports of the sensor. An electrically conductive connector port of an electronic device may also be attached to the clip in similar manner. Although two types of clips 1610 and 1710 are described herein, other type clips may be used to attached the bus of a wearable health monitoring harness to sensors and/or electronic devices.

Referring back to FIG. 15, the figure includes two operational stages 1501 and 1502. In stage 1501, the fabric 1210 may not be stretched. In stage 1502, the fabric 1210 may be stretched, for example, when the wearable health monitoring harness 1200 is worn by a person or is otherwise stretched. In stage 1502, as the fabric 1210 and the grooves 1241-1242 are stretched, the wires 1221-1222 may also be stretched, preventing damage and stress to the wires 1221-1222.

The clips, in some embodiments, may be covered by a layer of non-conductive stretchable material to prevent electrical shorts, to prevent electrical contacts with a person's body, and/or to protect a person's skin from any rough edges of the clips and/or clamps. FIG. 18 is top view of the wearable health monitoring harness of FIG. 15 where the clips are covered by a non-conductive stretchable material, according to various aspects of the present disclosure.

With reference to FIG. 18, the harness 1200 may include the stretchable non-conductive fabric covers 1811 and 1812 for covering the clips 1511 and 1512, respectively. For example, and without limitations, the stretchable non-conductive fabric covers 1811 and 1812 may be made of the same stretchable non-conductive fabric 1210 that is used for the wearable health monitoring harness 1200. The stretchable non-conductive fabric covers 1811 and 1812 may extend to both sides of the harness 1200 to cover the top and the bottom of the clips. In FIG. 18, the top surface of the stretchable non-conductive fabric covers 1811 and 1812 that covers the clips 1511-1512 is conceptually shown by the diagonal lines 1820.

FIG. 18, as shown, includes two operational stages 1801 and 1802. In stage 1801, the fabric 1210 may not be stretched. In stage 1802, the fabric 1210 may be stretched, for example, when the wearable health monitoring harness 1200 is worn by a person or is otherwise stretched. In stage 1802, as the fabric 1210, the grooves 1241-1242, and the covers 1811 and 1812 are stretched, the wires 1221-1222 may also be stretched, preventing damage and stress to the wires 1221-1222.

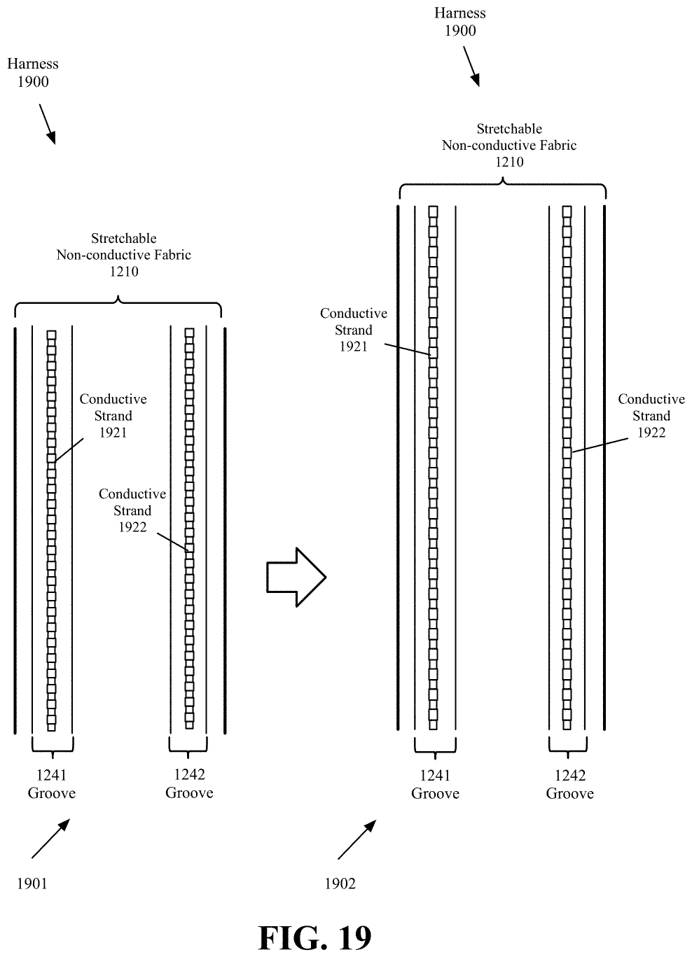

Some embodiments may use a stretchable conductive fabric for the bus wires. FIG. 19 is a top view of a portion of an example wearable health monitoring harness 1900 that includes a bus with stretchable conductors 1921-1922, according to various aspects of the present disclosure. With reference to FIG. 19, the stretchable non-conductive fabric 1210 and the grooves 1241-1242 may be similar to the corresponding components of FIG. 12.

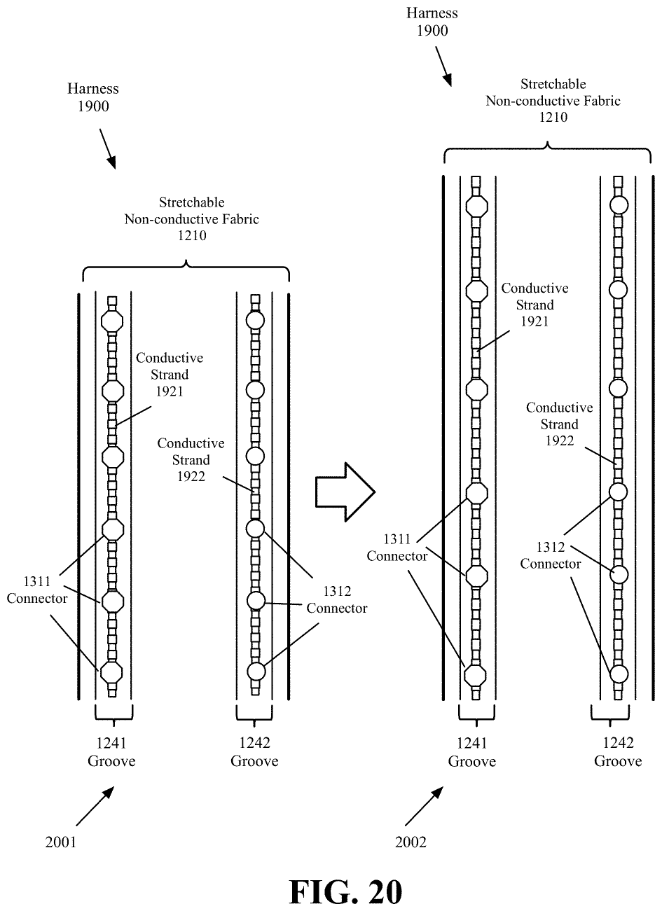

Different embodiments may provide different connectors, such as snap connectors, clips, clamps, etc., for connecting electronic devices, such as the controller, the sensor units, displays, audio/video devices, etc., to the bus of FIG. 19. These embodiments are described below with reference to FIGS. 20-22. For clarity, the connectors are not shown in FIG. 19 in order to illustrate how the stretchable conductors 1921-1922 may be stretched when the fabric 1210 is stretched.

With further reference to FIG. 19, the stretchable conductors may be in the shape of stretchable conductive strands 1921-1921, which may be made of stretchable electrically conductive fabric. The stretchable conductive strands 1921 and 1921, in some embodiments, may be in the shape of flexible cylindrical cords. The stretchable conductive strands 1921 and 1921, in some embodiments, may be made of conductive fabric, with metal strands woven into the construction of the fabric or by metal-coated yarns. The conductive fabric, in some embodiments, may include a non-conductive substrate, which is coated or embedded with electrically conductive elements. One example of the stretchable conductive fabric is the conductive Lycra fabric.

In some embodiments, narrow strands 1921-1922 of stretchable conductive fiber may be placed in the grooves 1241-1242 to act as conductive wires of the harness's bus. The wearable health monitoring harness 1900 may include flaps (not shown) to cover the grooves 1241-1242 after the stretchable conductive fabric strands 1921-1922 are installed in the grooves 1241-1242. The flaps may be made of the stretchable material as the stretchable fabric 1210. One side of each flap may be attached to one side of a groove 1241-1242. The flap may fit snuggly inside the groove to prevent the wire to come out of the groove and/or to prevent damage to the wire. A portion of the flap may be pushed aside to expose a pair of connectors (e.g., a pair of connectors 1311-1312 of FIG. 20) in the groove in order to connect an electronic device, such as a sensor unit, or the controller, to the pair of connectors.

With continued reference to FIG. 19, the figure as shown, includes two operational stages 1901 and 1902. In stage 1901, the fabric 1210 is not stretched. In stage 1902, the fabric 1210 may be stretched, for example, when the wearable health monitoring harness 1900 is worn by a person or is otherwise stretched. In stage 1902, as the non-conductive fabric 1210 and the grooves 1241-1242 are stretched, the stretchable conductive strands 1921-1922 may also be stretched, preventing damage and stress to the conductive strands 1921-1922.

The wearable health monitoring harness, in some embodiments, may include connectors (e.g., the connectors 131-134 of FIGS. 1A-2B) for connecting electronic devices such as the controller, the sensor units, displays, audio/video devices, etc., to the bus. FIG. 20 is top view of a portion of an example wearable health monitoring harness that includes a bus with stretchable conductors and a set of connectors for connecting electronic devices to the bus, according to various aspects of the present disclosure.

With reference to FIG. 20, the stretchable fabric 1210, the grooves 1241-1242, and the conductive strands 1921-1922 may be similar to the corresponding components of FIG. 19. FIG. 20 further illustrates a set of connectors 1311-1312 that are connected to the conductive strands 1921-1922, respectively. The connectors 1311 may, for example, be similar to the connectors 131 or 133 of FIGS. 1A-2B. The connectors 1312 may, for example, be similar to the connectors 132 or 134 of FIGS. 1A-2B. The connectors 1311-1312 of FIG. 20 may provide similar functionalities as the corresponding connectors 1311-1312 of FIG. 13, which were described above.

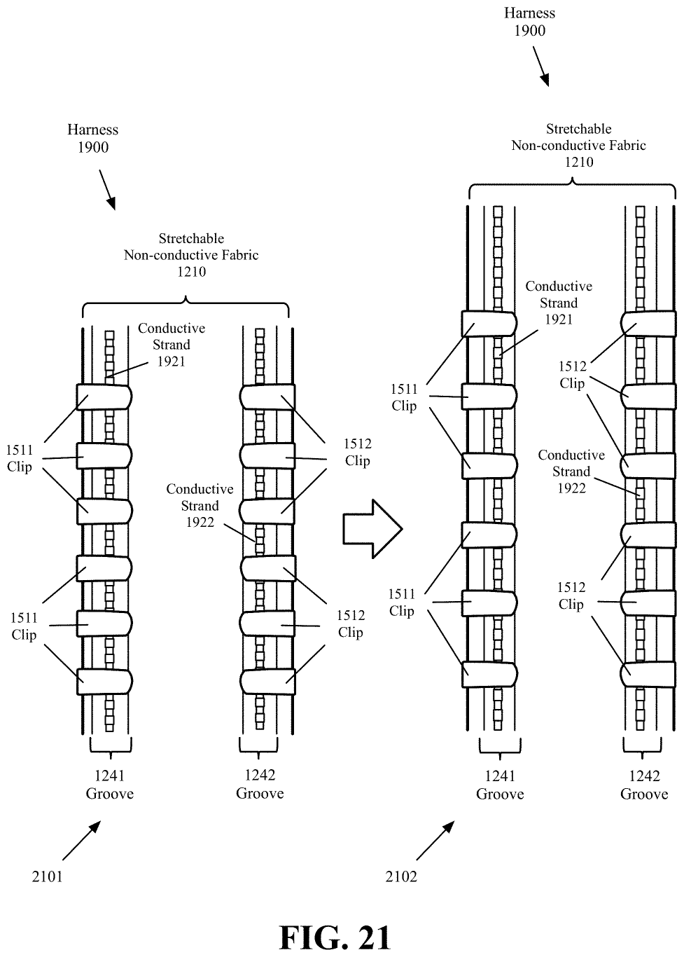

The wearable health monitoring harness, in some embodiments, may include clips and/or clamps for connecting electronic devices such as the controller, the sensor units, displays, audio/video devices, etc., to the bus. FIG. 21 is top view of a portion of an example wearable health monitoring harness a with bus with stretchable conductors and a set of clips for connecting electronic devices to the bus, according to various aspects of the present disclosure.

With reference to FIG. 21, the stretchable fabric 1210, the grooves 1241-1242, and the conductive strands 1921-1922 may be similar to the corresponding components of FIGS. 19 and 20. FIG. 21 further illustrates a set of clips 1511-1512 that are connected to the conductive strands 1921-1922, respectively. The clips 1511 may, for example, function similar to the connectors 131 or 133 of FIGS. 1A-2B. The clips 1512 may, for example, function similar to the connectors 132 or 134 of FIGS. 1A-2B. Although several examples are described herein with reference to clips, some embodiments may use clamps to achieve similar functionalities as the clips.

With further reference to FIG. 21, the clips 1511-1512 may provide the same functionalities as the clips 1511-1512 of FIG. 15, which was described above. FIG. 21, as shown, includes two operational stages 2101 and 2102. In stage 2101, the fabric 1210 may not be stretched. In stage 2102, the fabric 1210 may be stretched, for example, when the wearable health monitoring harness 1900 is worn by a person or is otherwise stretched. In stage 2102, as the fabric 1210 and the grooves 1241-1242 are stretched, the conductive strands 1921-1922 may also be stretched, preventing damage and stress to the conductive strands 1921-1922.

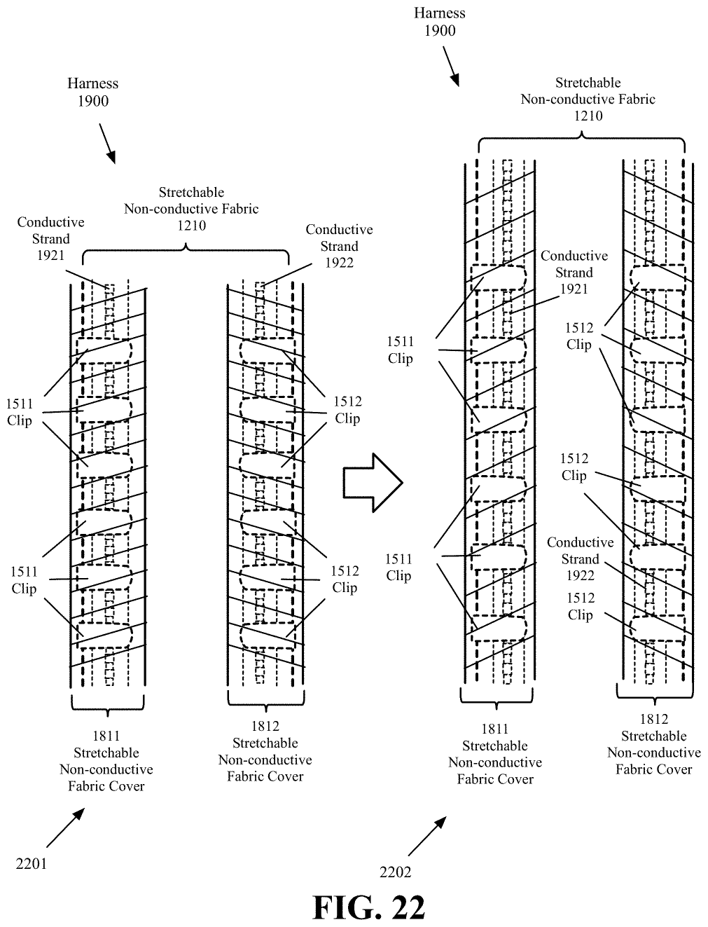

The clips, in some embodiments, may be covered by a layer of non-conductive stretchable material to prevent electrical shorts, to prevent electrical contact with a person's body, and/or to protect the person's skin from any rough edges of the clips and/or clamps. FIG. 22 is top view of the wearable health monitoring harness of FIG. 21 where the clips are covered by a non-conductive stretchable material, according to various aspects of the present disclosure.

With reference to FIG. 22, the harness 1900 may include the stretchable non-conductive fabric covers 1811 and 1812 for covering the clips 1511 and 1512, respectively. The stretchable non-conductive fabric covers 1811 and 1812 of FIG. 22 may function similar as the corresponding components of FIG. 18.

FIG. 22, as shown, includes two operational stages 2201 and 2202. In stage 2201, the fabric 1210 may not be stretched. In stage 2202, the fabric 1210 may be stretched, for example, when the wearable health monitoring harness 1900 is worn by a person or is otherwise stretched. In stage 2202, as the fabric 1210, the grooves 1241-1242, and the covers 1811 and 1812 are stretched, the conductive strands 1921-1922 may also be stretched, preventing damage and stress to the conductive strands 1921-1922.

In should be noted that the embodiments described above with reference to FIGS. 13 and 20, in some embodiments, may also include stretchable non-conductive fabric covers similar to the stretchable non-conductive fabric covers 1811-1812 of FIGS. 18 and 22 to prevent electrical shorts, to prevent electrical contacts with a person's body, and/or to protect a person's skin from any rough edges of the snap connectors and/or pin connectors of the harness.

Although several examples were described above with reference to the wires 1221-1222 and/or the conductive strands 1921-1922 being placed inside the grooves 1241-1242, respectively, some embodiments may not include the grooves 1241-1242. In these embodiments, the wires 1221-1222 and/or the conductive strands 1921-1922 may be guided to run across the wearable health monitoring harness by a set of pins, clips, etc., that may loosely hold the wires 1221-1222 and/or the conductive strands 1921-1922 on the harness and at the same time allow for the stretching of the sawtooth shaped wires and/or the stretchable conductive stands.

In some embodiments, a display unit and/or an audio/video unit that includes a display may be attached to the wearable health monitoring harness. The display, in some embodiments, may be an energy efficient display. For example, and without limitations, the display may be a reflective (e.g., liquid crystal display (LCD), E Ink (or electronic ink), Rdot, etc.) or transflective display. The display, in some embodiments, may be a flexible display to prevent damage while the wearable health monitoring harness is worn by a person. The display, in some embodiments, may be a light emitting diode (LED) display and/or may include a set of LED lights.

FIG. 23 is a schematic front view of an audio/video device that may be attached to a wearable health monitoring harness, according to various aspects of the present disclosure. With reference to FIG. 23, the audio/video device 2300 may include one or more microphones 2325, a display 2330, one or more speakers 2330, and and/or an on/off switch 2340.

The display may be an energy efficient display as described above. The audio/video device 2300 may be used by the controller 100 (FIG. 4) to display the health status 2375 of different sensor units, the battery level 2370, one or more vital signals 2360 of a person as being recorded by the sensor units, and/or display graphs (not shown), such as for example, and without limitations, the ECG graphs of the person. The display may include the scroll tools 2350-2355 to allow scrolling additional data into the display area.

The audio/video device 2300 may be used, for example, and without limitations, to record the surrounding sounds, play recorded instructions and/or play live conversations with remote care givers and operators. Some embodiments may provide the display 2330 without the microphone(s) 2325 and/or the speaker(s) 2330.

In some embodiments, the controller 100 (FIG. 4) may send the health status sensor units, the battery level, the vital signals of the person wearing the harness, and/or the graphs of the vital signs to a display that is external to the wearable health monitoring harness. FIG. 24 is a schematic front view of an electronic device that may receive and display information from the controller of a wearable health monitoring harness, according to various aspects of the present disclosure. With reference to FIG. 24, the electronic device 2400 may be a mobile device, such as a smartphone, a tablet, a laptop, etc. The electronic device 2400 may also be a computing device such as, a desktop computer, a server, etc. The electronic device 2400 may be associated with the person who is wearing the harness, with a hospital or clinic, with a physician, a nurse, a caregiver, a researcher, etc.

With further reference to FIG. 24, a user interface (UI) 2440 may be displayed on the display 2420 of the electronic device 2400. The UI 2440 may display different information received from the wearable health monitoring harness. In the example of FIG. 24, the UI 2440 may display the health status 2375 of different sensor units, the battery level 2370 of the wearable health monitoring harness, one or more vital signals 2360 of the person as being recorded by the sensor units, and/or display graphs (not shown), such as for example, and without limitations, the ECG graphs of the person.

The UI 2440 may include scroll tool 2455-2460 to allow scrolling additional data into the display area. The UI 2440 may further display data received from the sensor units attached to the wearable harness of the present embodiments. The displayed sensor data may be used, for example, and without limitations, for finetuning the position of the sensor units on the wearable harness. For example, a sensor unit may be attached to a pair of connectors of the harness, the displayed sensor data may be examined, and the sensor unit may be detached from the pair of connectors and re-attached to a different pair of connectors. This process may be repeated and the sensor unit may be connected to different pairs of connectors until the data received from the sensor unit satisfies one or more criteria. A similar process may be performed to finetune the position of the sensor units by displaying the sensor data on a display unit and/or on a display of an audio/video unit that is attached to the harness.

The controller 100 (FIG. 4), in some embodiments, may send a warning message when one of the sensor units fail to operator, the charge level of the battery 420 goes below a threshold, one or more vital signs exceeds (or go below) a limit, etc. FIG. 25 is a schematic front view of an electronic device that may receive a warning message from the controller of a wearable health monitoring harness, according to various aspects of the present disclosure. With reference to FIG. 25, the electronic device 2500 may be an audio/video device or a display device attached to the wearable health monitoring harness. The electronic device 2500 may be an external electronic device such as, for example, and without limitations, a mobile device, a desktop computer, a server, etc.

In the example of FIG. 25, the electronic device 2500 may be a smartphone. FIG. 25, as shown, includes three operational stages. In stage 2501, the mobile device may display information 2505 unrelated to the wearable health monitoring device. As shown, the electronic device 2500 may receive a notification 2520 that a status message from the health monitoring harness has received.

In some aspects of the present embodiments, the notification 2520 may be displayed on the display 2590 of the electronic device 2500 when the electronic device 2500 is in a locked mode. In some aspects of the present embodiments, a device is in the locked mode when only a reduced set of controls can be used to provide input to the device. In some aspects of the present embodiments, when the display 2590 of the electronic device 2500 is turned off (e.g., to save battery power), the electronic device 2500 may turn on the display 2590 and may display the notification 2520.

In stage 2502, the notification 2520 may be selected to open an application through which the notification is received (e.g., an application that is associated with the wearable health monitoring harness) and to view the message associated with the notification 2520. In response to the selection of the notification 2520, the application 2560 associated with the wearable health monitoring harness may be opened in stage 2503.

As shown, one or more warnings and/or messages 2525 may be displayed on the display 2590 of the electronic device 2500. The warnings and/or messages may be used by medical person and/or the person wearing the harness to adjust and/or replace faulty sensor units, to remove sources of noise and interference, to provide medical assistance to the person wearing the harness, etc.

The electronic devices described above may include memory. The memory may be one or more units of similar or different memories. For example, the electronic devices' memory may include, without any limitations, random access memory (RAM), read-only-memory (ROM), read-only compact discs (CD-ROM), erasable programmable read-only memories (EPROMs), electrically erasable programmable read-only memories (EEPROMs), flash memory (e.g., secured digital (SD) cards, mini-SD cards, micro-SD cards, etc.), magnetic and/or solid state hard drives, ultra-density optical discs, any other optical or magnetic media, and floppy disks.

The electronic devices described above may include one or more processing units. For example, the processing unit(s) in above examples may be single-core processor(s) or multi-core processor(s) in different embodiments. The electronic devices in some of the present embodiments may store computer program instructions in the memory, which may be a machine-readable or computer-readable medium (alternatively referred to as computer-readable storage medium, machine-readable medium, or machine-readable storage medium).

Many of the above-described features and applications may be implemented as software processes (or programs) that are specified as a set of instructions recorded on a computer readable storage medium. The computer-readable medium may store a program that is executable by at least one processing unit and includes sets of instructions for performing various operations. Examples of programs or computer code include machine code, such as is produced by a compiler, and files including higher-level code that are executed by a computer, an electronic component, or a microprocessor using an interpreter. From these various memory units, the processing unit may retrieve instructions to execute and data to process in order to execute the processes of the present embodiments.

As used in this disclosure and any claims of this disclosure, the terms such as “processing unit,” “processor,” “controller,” “microcontroller,” “server”, and “memory” all refer to electronic or other technological devices. These terms exclude people or groups of people. For the purposes of this disclosure, the terms display or displaying means displaying on an electronic device. As used in this disclosure and any claims of this disclosure, the terms “computer readable medium,” “computer readable media,” and “machine readable medium” are entirely restricted to tangible, non-transitory, physical objects that store information in a form that is readable by a processing unit. These terms exclude any wireless signals, wired download signals, and any other transitory and ephemeral signals. As used in this disclosure and any claims of this disclosure, the term application is referred to an application program (or a program) that performs a set of tasks.

The above description presents the best mode contemplated for carrying out the present embodiments, and of the manner and process of practicing them, in such full, clear, concise, and exact terms as to enable any person skilled in the art to which they pertain to practice these embodiments. The present embodiments are, however, susceptible to modifications and alternate constructions from those discussed above that are fully equivalent. Consequently, the present invention is not limited to the particular embodiments disclosed. On the contrary, the present invention covers all modifications and alternate constructions coming within the spirit and scope of the present disclosure. For example, the steps in the processes described herein need not be performed in the same order as they have been presented, and may be performed in any order(s). Further, steps that have been presented as being performed separately may in alternative embodiments be performed concurrently. Likewise, steps that have been presented as being performed concurrently may in alternative embodiments be performed separately.

What is claimed is:

1. A wearable health monitoring harness, comprising:
a vest;
a bus configured to carry digitized signals and power, the bus comprising first and second wires running across the vest;
first and second plurality of connectors, the first plurality of connectors connected to the first wire of the bus, the second plurality of connectors connected to the second wire of the bus, the first and second plurality of connectors configured to form a plurality of pairs connectors, each pair of connectors comprising a connector from the first plurality of connectors and a connector from the second plurality of connectors;
a battery comprising first and second ports, the first battery port connected the first wire of the bus, and the second battery port connected the second wire of the bus;
a set of one or more sensor units, each sensor unit comprising:
sensor circuitry for measuring one or more bodily signals of a person wearing the vest;
an analog to digital converter (A/D) configured to:
receive analog signals from the corresponding sensor circuitry; and
convert the received analog signals to digitized data;
a processor configured to communicate data over the bus; and
a pair of connector ports for detachably connecting to a pair of the connectors in the plurality of pairs of connectors;
a controller unit comprising:
a transceiver;
a processor configured to:
receive digitized data from the set of sensor units' processors over the bus; and
send the digitized data to one or more external devices using the transceiver.

2. The wearable health monitoring harness of claim 1, wherein the plurality of pairs of connectors are configured to allow finetuning a position of the sensor units by attaching a sensor unit to a pair of connectors, examining the sensor unit's digitized data, detaching the sensor unit from the pair of connectors when the digitized data of the sensor unit does not satisfy one or more criteria, re-attaching the sensor unit to a different pair of connectors, and repeating the attaching, the examining, the detaching, and re-attaching until the sensor unit's digitized data satisfies said one or more criteria.

3. The wearable health monitoring harness of claim 1,
wherein the vest is made, at least partially, of a stretchable fabric,
wherein the vest comprises first and second grooves,
wherein the first and second wires of the bus are placed in a sawtooth or wavy shape in the first and second grooves, respectively, and
wherein the sawtooth or wavy shaped first and second wires of the bus are configured to stretch inside the corresponding grooves when the fabric of vest is stretched.

4. The wearable health monitoring harness of claim 1,
wherein the vest is made, at least partially, of a stretchable fabric,
wherein the vest comprises first and second grooves,
wherein the first and second wires of the bus are made, at least partially, from a stretchable conductive fabric and are placed in the first and second grooves, respectively, and
wherein the first and second wires of the bus are configured to stretch when the fabric of the vest is stretched.

5. The wearable health monitoring harness of claim 1, wherein the sensor circuitry of the sensor units comprise one or more of electrocardiogram (ECG) sensors, body temperature sensors, oxygen sensors, motion sensors, breathing rate sensors, impedance plethysmography sensors, blood pressure sensors, and microphones.

6. The wearable health monitoring harness of claim 1, wherein the sensor units comprise a breathing rate sensor unit measuring the breathing rate of the person wearing the vest, the breathing rate sensor unit comprising:
a stretchable conductive fabric configured to conduct electricity and change electrical resistance when stretched,
wherein the processor of the breathing rate sensor unit is configured to calculate the breathing rate of the person based on a change in the electrical resistance of the stretchable conductive fabric of the breathing rate sensor unit.

7. The wearable health monitoring harness of claim 1, wherein the first and second plurality of connectors comprise one or more of snap connectors, pins, clamps, and clips.

8. The wearable health monitoring harness of claim 7 further comprising first and second non-conductive stretchable fabrics configured to at least partially cover the first and second plurality of connectors of the wearable health monitoring harness, respectively.

9. The wearable health monitoring harness of claim 1, further comprising a display unit configured to removably attach to a connector pair in the plurality of connector pairs, the processor of the controller unit configured to display, on the display unit, one or more of the health status of more or more of the sensor units, the digitized data received from more or more of the sensor units, and one or more warning messages related to the bodily signals of the person wearing the vest.

10. The wearable health monitoring harness of claim 1, further comprising an audio/video unit configured to removably attach to a connector pair in the plurality of connector pairs, the audio/video unit configured to:
record surrounding sounds;
play recorded instructions;
play live conversations with a remotely located person; and
display one or more of the health status of more or more of the sensor units, the digitized data received from more or more of the sensor units, and one or more warning messages related to the bodily signals of the person wearing the vest.

11. The wearable health monitoring harness of claim 1, wherein the controller unit comprises said battery.

12. The wearable health monitoring harness of claim 1, wherein at least one sensor unit in the set of sensor units comprise memory,
wherein the processor of the controller unit is configured to send one or more signals over the bus to the processor of the at least one sensor unit to send digitized data to the processor of the controller unit; and
wherein the processor of the at least one sensor unit is configured to:
store the digitized data received from the A/D of the sensor unit in the memory of the sensor unit;
receive said one or more signals from the processor of the controller unit over the bus to send the digitized data to the processor of the controller unit; and
in response to receiving said one or more signals, send the digitized data to the processor of the controller unit.

13. The wearable health monitoring harness of claim 1, wherein the controller unit comprise memory, wherein the processor of the controller unit is configured to:
process the digitized data received from one or more of the sensor units to perform one or more of filtering the digitized data, comparing the digitized data to one or more limits, generating one or more reports, generating one or more alerts, performing calculation on the digitized data, and filtering the digitized data; and
store the digitized data received from the sensor units and the processed digitized data in the memory of the controller unit.

14. The wearable health monitoring harness of claim 1, wherein the processor of the controller unit is configured to:
receive one or more signals from an electronic device external to the wearable health monitoring harness; and
in response to receiving the one or more signals from the electronic device, send digitized sensor data associated with at least one sensor unit to the electronic device.

15. The wearable health monitoring harness of claim 1,
wherein the plurality of pairs of connectors comprises a set of two or more pairs of connectors configured to attach to a surface of the vest that touches the body of the person wearing the vest, and
wherein the set of sensor units comprise one or more sensor units comprising a surface configured to being in flat contact with the body of the person when the sensor unit is attached to a pair of connectors in the set of connector pairs.

16. The wearable health monitoring harness of claim 1,
wherein the plurality of pairs of connectors comprises a set of two or more pairs of connectors configured to attach to a surface of the vest that does not touch the body of the person wearing the vest, and
wherein the set of sensor units comprise one or more sensor units configured to attach to a pair of connectors in the set of connector pairs.

17. The wearable health monitoring harness of claim 1,
wherein the vest comprises first, second, and third straps, wherein the first strap is configured to be worn around the chest of the person wearing the vest,
wherein the second and third straps are configured to be worn around the shoulders of the person, and
wherein the second and third straps are connected to the first strap at a plurality of locations.

18. The wearable health monitoring harness of claim 1, wherein the vest comprises one or more fasteners for adjusting a size of the vest.

19. The wearable health monitoring harness of claim 1,
wherein the vest comprises first and second straps and a back section,
wherein the back section is configured to cover a portion of the upper back of the person wearing the vest and connects to the first and second straps in a vicinity of person's shoulder area, and
wherein the first and second straps each goes over a shoulder of the person, cross each other over the chest of the person, and join each other with a fastener on the back of the person.

20. The wearable health monitoring harness of claim 1,
wherein the vest comprises first, second, and third straps,
wherein the first strap is configured to form an arc section on the upper back and shoulders of the person wearing the vest, to form an X shape in front of the chest of the person, and to go around the torso of the person to form a back section in the back of the person, and
wherein the second and the third straps are configured to connect the arc section of the first strap to the back section of the first strap.
